SEQUENCE LISTING (2 sequences)

SEQ ID NO: 1 - protein, 187 aa, Homo sapiens
MPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGK

SEQ ID NO: 2 - protein, 94 aa, Artificial sequence
  Consensus amino acid sequence characterising an RKIP motif
TLXXXDPDXPXXXXXXXXEXXHXXXXXXXXXXXXXXXXXXXXXXXXXXXXXXXXXXXXXXXXXXXXXXXXXXYXXXXPXXXXGXHRXVXXXXXQ

(19) United States
(12) Patent Application Publication
(10) Pub. No.: US 2009/0215038 A1
(43) Pub. Date: Aug. 27, 2009

(54) Prediction of Metastasis

(76) Inventors: Fahd Al-Mulla, Safat (KW); Suzanne Hagan, Lanarkshire (GB); Walter Kolch, Lanarkshire (GB)

Correspondence Address: MORGAN LEWIS & BOCKIUS LLP, 1111 PENNSYLVANIA AVENUE NW, WASHINGTON, DC, 20004, US

(21) Appl. No.: 11/908,120

(22) PCT Filed: Mar. 7, 2006
(86) PCT No.: PCT/GB06/00807

§ 371 Date: Jun. 16, 2008

Related U.S. Application Data
(60) Provisional application No. 60/659,123, filed on Mar. 7, 2005.

(30) Foreign Application Priority Data
Oct. 19, 2005 (GB) 0521286.5

(51) Int. Cl. C12Q 1/68; G01N 33/574; C12M 1/34

(52) U.S. Cl. 435/6; 435/7.23; 435/287.2

(57) ABSTRACT

The present invention relates to the prediction of metastasis by determining RKIP expression levels, and to products and kits adapted for such methods. In some embodiments, the present invention relates to methods of assigning a treatment or monitoring regime to a cancer patient according to the level of RKIP expression.

### FIELD OF THE INVENTION

The present invention relates to the prediction of metastasis by determining RKIP expression level, and to products and kits adapted for such methods.

### BACKGROUND

The majority of cancer deaths are associated with metastatic disease. For instance, in colorectal cancer (CRC), almost one fourth of early stage patients with non-metastatic disease relapse with metastasis later. Patients diagnosed with metastatic forms of CRC, namely stages Dukes' C with lymph node metastasis and Dukes' D with haematological dissemination, have five year survival rates of 37% and 11%, respectively. Patients diagnosed at an early stage (Dukes' A and B) with no evidence of metastatic disease at time of surgery have a significantly better prognosis featuring five year survival rates of 85% and 67%, respectively (Cancer Research UK, 2004). However, a significant proportion of these patients (10-45%) relapse with metastatic disease (Compton, 2003; Kahlenberg et al., 2003; Ovaska et al., 1990; Olson et al., 1980).

Chemotherapy has been clearly proved effective for Dukes' C stage tumours reducing recurrence between 170-33%. Newer studies also indicate the value of chemotherapy for some patients with early colorectal cancer at risk of metastatic relapse (Andre and de Gramont, 2004). However, although chemotherapeutic intervention has been implemented for some patients with early colon cancer in several centres, its benefits as a routine treatment have been questioned (Buyse and Piedbois, 2001; Marsoni, 2001). The side effects associated with the treatment make it desirable to avoid application of the treatment except in cases of high relapse risk.

Thus, the population at risk of relapse could benefit from close monitoring and/or early adjuvant chemotherapy. However, suitable markers for identifying this population are currently lacking.

Recent evidence showed that the retention of heterozygosity at chromosomes 17p (at the p53 gene locus) or 18q sites, in particular D18S61, could identify Dukes' B and C stage patients that would benefit from adjuvant chemotherapy (Barratt et al., 2002). These data indicate the potential value for the use of molecular markers as predictors in treatment decisions. Further attempts to identify prognostic markers have ranged from assessing specific histopathological features, to molecular methods such as counting alleles and detection of micrometastases by RT-PCR (Petersen et al., 2002; Zhou et al., 2002; Liefers et al., 1998). In addition, metastasis suppressor genes (including nm23, E-cadherins, CD44 and KAI1) have been investigated in colorectal cancer, but their value in predicting metastatic relapse in early colorectal cancers and in discriminating between risk and non-risk populations so as to be useful in a clinical situation has been mixed (Lombardi et al., 1999; Hartsough and Steeg, 1998; Heys et al., 1998; Dorudi et al., 1995; Mulder et al., 1994).

RKIP was isolated as a protein that could interfere with the activation of the Raf-MEK-ERK pathway by inhibiting the phosphorylation and activation of MEK by Raf-1 (Yeung et al., 2000; Yeung et al., 1999b). Subsequently, RKIP also was shown to suppress the activation of NFκB by blocking the inactivation of IκB, the inhibitor of NFκB (Yeung et al., 2001). Both pathways play an important role in cancer and invasion (Greten and Karin, 2004; Reddy et al., 2003). More recently, RKIP has been identified as a metastasis suppressor gene in several cancer cells lines including those of prostate cancer (Fu et al., 2003; Chatterjee et al., 2004), breast cancer (Chatterjee et al., 2004) and melanoma (Schuierer et al., 2004).

These studies show a reduction or loss of RKIP protein expression in metastatic cell lines or metastatic lesions. The reconstitution of RKIP protein levels in metastatic cell lines by exogenous expression impaired in vitro invasiveness (Schuierer et al., 2004) and the ability to form metastases in mouse models (Fu et al., 2003). In contrast, downregulation of RKIP expression by antisense RNA promoted invasiveness (Schuierer et al., 2004). In an orthotopic mouse model for prostate cancer RKIP expression did not affect primary tumour growth despite preventing the tumour from metastasizing (Fu et al., 2003). These results indicate that RKIP is not a tumour suppressor gene, but rather a suppressor of metastasis. Furthermore, RKIP expression has been shown to sensitize prostate cancer cells to apoptosis induced by DNA damaging drugs by interfering with the Raf-MEK-ERK and NFκB pathways (Chatterjee et al., 2004).

### SUMMARY OF THE INVENTION

The present inventors have now found that the level of RKIP expression in a primary tumour is a predictor of metastasis. A strong correlation between RKIP expression in primary tumours having no detectable metastasis and disease free survival after treatment of that primary tumour has been shown. Low levels of RKIP expression in a tumour indicate a real risk of tumour metastasis. Conversely, high levels of RKIP in a primary tumour indicate a low risk of tumour metastasis. It is therefore believed that RKIP is clinically relevant as a prognostic marker in primary tumours. The knowledge of a patient's risk of experiencing metastasis and relapse is very important and can assist in deciding the most appropriate treatment for the patient.

Thus, the present invention relates to RKIP as a prognostic marker in cancer, particularly in cancer having no detectable metastasis. The invention also provides materials and methods for assigning a treatment and/or or monitoring regime based on the expression levels of RKIP in a tumour sample from a patient. The invention further provides materials and methods for predicting development of metastasis by determining RKIP expression. The cancer is preferably colorectal cancer.

In one aspect, the present invention provides a method of assigning a treatment and/or monitoring regime to a cancer patient, comprising:
determining the RKIP expression level in a sample from a primary tumour obtained from the patient;
assigning a treatment and/or monitoring regime to the patient according to the RKIP expression level.

The treatment and/or monitoring regime referred to in the present application may be for treatment or monitoring of metastasis. Preferably it is for preventative treatment of metastasis or monitoring of development of detectable metastasis in a patient assessed as not having detectable metastasis, e.g., in a patient who has received treatment of the primary tumour. The treatment and/or monitoring regime may be applied in addition to, and preferably after, the treatment of the primary tumour. It may be an adjuvant regime.

The patient referred to in the present application has preferably been assessed as not having detectable metastasis, e.g., prior to assignment of the treatment or monitoring regime. It is preferred that the patient is assessed as having had no metastasis at the time of receiving treatment for the primary tumour, e.g., by surgery. Thus, the primary tumour may have been assessed as non-metastatic. It may be Dukes' stage A or B colorectal cancer, or a cancer having a staging indicating a non-metastatic cancer in another staging system.

The method may comprise classifying the RKIP expression level in order to assign the treatment regime. This classification may be done by comparing the RKIP expression level to previously obtained RKIP levels. Classification may be according to risk of relapse.

In another aspect, the invention provides a method of classifying the expression level of RKIP in a sample from a primary tumour from a patient, in order to assign a treatment regime based on said expression levels (which may be a regime as described above), the method comprising determining the level of expression of RKIP, and classifying the level of expression. The classification may be based on previously obtained RKIP levels, e.g., in tumour samples (particular tumour samples of the same type as the cancer in question and preferable colorectal cancer samples), or on normal tissue such as normal epithelial mucosa or normal endothelial cells of blood and lymphatic vessels. Having classified the expression level, the method may optionally further include the step of assigning a regime of monitoring or treatment based on the classification.

A previously obtained RKIP expression level may be a level in primary tumour, preferably of the same tissue type as the primary tumour of the patient e.g., a colorectal tumour. It may be a level in a primary tumour associated or not associated with metastasis, or in non-metastasised primary tumours from patients who go on or do not go on to develop metastasis. The classification may be carried out by comparison to a set of references as discussed further below.

The number of possible classes and the number of possible regimes assigned may be two, three or four or more, but it may be preferred in some embodiments to have only two classes for clinical certainty.

It is preferred that the samples are classified according to risk of developing detectable metastasis, more preferably of metastatic recurrence in a patient who has received treatment for a primary tumour and has been assessed as not having detectable metastasis, e.g., at the time of treatment of that primary tumour. For example, high RKIP expression may indicate low risk, and low RKIP expression may indicate high risk of metastasis. There may also be a class in which moderate RKIP expression indicates moderate risk.

In another aspect, the invention provides a method of predicting metastasis in a patient, comprising determining the RKIP expression level in a sample from a primary tumour obtained from the patient.

The method may be a method of predicting a risk of developing detectable metastases. The method may comprise classifying the RKIP expression according to the level of risk based on previously obtained RKIP expression levels in tumour samples as described above. The method may be of predicting metastatic recurrence occurring after treatment of the primary tumour.

In the methods of the present invention, determining the RKIP expression level may be done indirectly, by determining the RKIP activity resulting from the expression. Alternatively, the levels of an RKIP expression product may be assessed directly, e.g., using a specific binding partner of that RKIP expression product.

In another aspect, the invention provides a kit for classifying the RKIP expression level in a sample of primary tumour obtained from a patient in order to assign a treatment and/or monitoring regime (which may be a regime such as described above) based on said expression level, wherein said kit comprises a specific binding partner for an RKIP expression product and a set of references against which the expression level can be compared in order to classify said level.

The specific binding partner may be labelled. The labelling may be indirect. For example, the kit may further comprise a second binding member which binds specifically to the first binding member, and this second binding member may be labelled.

The invention may also provide a kit for classifying the RKIP expression level in a sample of primary tumour obtained from a patient in order to assign a treatment and/or monitoring regime (which may be a regime such as described above) based on said expression level, wherein the kit comprises a specific binding partner for an RKIP expression product, and also comprises a specific binding partner for the expression product of another gene, for example, a prognostic marker or another predictor of metastasis in cancer, particularly in colorectal cancer. The at least one other expression product may in some embodiments be selected from carcinoembryonic antigen (CEA), lipid associated sialic acid (LASA) CA19-9, p53, Ki-Ras or Raf mutations, Nm23, E-cadherin, CD44, and/or KAI1. They may also be selected from p53, VEGF, EGFR, ER (estrogen receptor), BRCA2, BRCA1, CA 15.3, c-erbB-2 and progesterone receptor (PR), particularly in breast cancer. The specific binding member may be labelled as described above.

In another aspect, the invention provides a method of establishing a set of references for classifying the RKIP expression level in a sample from a primary tumour in order to assign a treatment regime based on the expression level (which may be a regime as described above), the method comprising:
determining the RKIP expression level in a sample from primary tumours obtained from a plurality of patients, wherein said patients have been assessed as having no detectable metastasis;
determining whether said patients develop detectable metastasis;
establishing a set of references for classifying the RKIP expression level which maximise the difference in risk of developing detectable metastasis between patients assigned to each class.

Preferably, said patients have received treatment for the primary tumour, and the determination as to whether said patients develop detectable metastasis is made after said treatment. The determination of metastatic relapse may be made after a period of less than 1, or 1, 2, 3, 4, 5, 6, 7, 8, 9, or 10 years, or more.

Determination of metastatic relapse may be made by any of the methods for detecting or monitoring metastasis described further below.

In a still further aspect, the invention provides an apparatus for classifying the RKIP expression level in a sample from a primary tumour in order to assign a treatment regime based on said expression level, the apparatus comprising;
a port for receiving the sample;
a detector for detecting the expression level of RKIP in said sample;
means for providing a set of references for classifying the RKIP expression level;
means for comparing the detected expression level to said references so as to classify said expression level; and
an output for providing the classification of the sample to the user.

The means for providing a set of references for classifying the RKIP expression level and the means for comparing the detected expression level to said references may be independently selected from software and hardware. Although referred to as separate means they may be the same, e.g., they may be the processes of a computer.

The detector may for example be a device for assessing the density of a label, where said label is specifically associated with an RKIP expression product.

The output may for example be a display screen, a printer for providing a printout, or an electronic output to another device which provides the classification of the sample to the user.

In another aspect, the invention provides in vitro use of a binding partner of an RKIP expression product for determining the risk of metastatic recurrence in a patient.

A significant relationship between the risk of tumour metastasis and RKIP expression levels has been shown by the inventors in colorectal cancer. In all aspects of the present invention it is preferred that the cancer is colorectal cancer. The strength of the relationship suggests a real clinical value in prognosis and shows that RKIP can be used as a predictor of metastasis. It is particularly preferred that the primary tumour from which the sample is obtained is a Dukes' A or B stage colorectal cancer.

In other embodiments it may be preferred that the cancer is breast cancer, or a cancer other than breast cancer, for example, of the lung, prostate, pancreas, brain, bladder, stomach, kidney, liver, gallbladder, ovary, testes, cervix, uterus, salivary/parotid glands, squamous epithelium, skin, bone and the like, or neuronal cancer (e.g., gliomas or astrocytomas).

It may be preferred in some embodiments that RKIP is the only protein whose level of expression is taken into account for the risk determination or for the assignment of a treatment or monitoring regime. (It is not intended that this should exclude assessment of the levels of other proteins as controls). It may be preferred in some embodiments that RKIP is used as the sole indicator, e.g., sole protein marker for metastatic relapse, in the above methods, kits or apparatus.

In other embodiments, it may be preferred that RKIP provides only one of a plurality of indicators or factors to be taken into account when assessing overall risk or when assigning a treatment or monitoring regime. Other indicators may be levels of expression of other proteins and/or clinical parameters such as tumour staging, tumour differentiation/grade, site and/or apoptotic index.

For example, carcinoembryonic antigen (CEA), lipid associated sialic acid (LASA) CA19-9, DNA ploidy, p53, Ki-Ras or Raf mutations, Nm23, E-cadherin, CD44, and/or KAI1 may be used, particularly in colorectal cancer.

Other indicators may be VEGF, tumour blood vessel counts and/or EGFR. While not clinically useful on their own in early stage colorectal cancer they may be useful in combination with RKIP expression levels. In breast cancer, it may be preferred that RKIP expression levels are measured together with ER status (breast cancer patients can be ER positive or negative), p53, BRCA2, BRCA1, CA 15.3, c-erbB-2 and/or progesterone receptor (PR).

The invention will now be further described, with reference to the accompanying figures, in which:

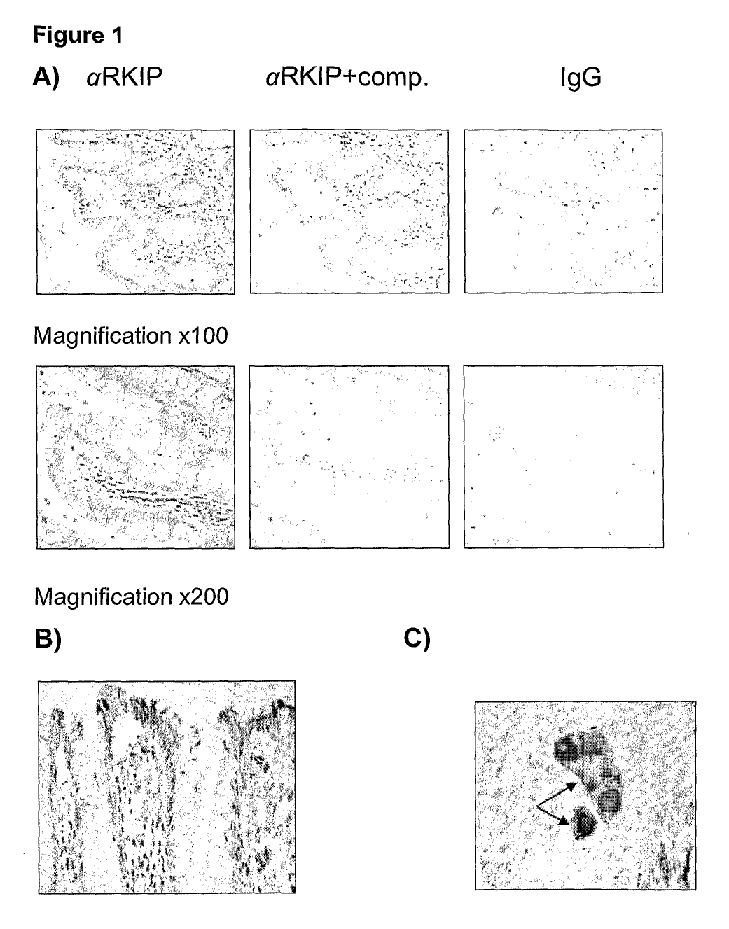

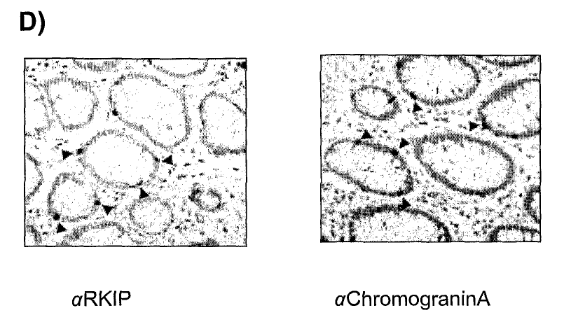

FIG. 1 shows Expression of RKIP in normal colon. (A) Specificity of the RKIP antibody. Adjacent sections of paraffin embedded colon tissue was stained with RKIP antibody (αRKIP), RKIP antibody preadsorbed with RKIP protein (αRKIP+comp.) or unrelated IgG as indicated. (B) Sagittal section through a colon crypt. RKIP expression increases as cells differentiate towards the top of the crypt. (C) RKIP staining of Auerbach's neural plexus, indicated by arrows. (D) Transverse section through crypts showing strong RKIP expression in neuroendocrine cells, indicated by arrowheads. These cells were identified by staining with antibody against the neuroendocrine marker chromograninA.

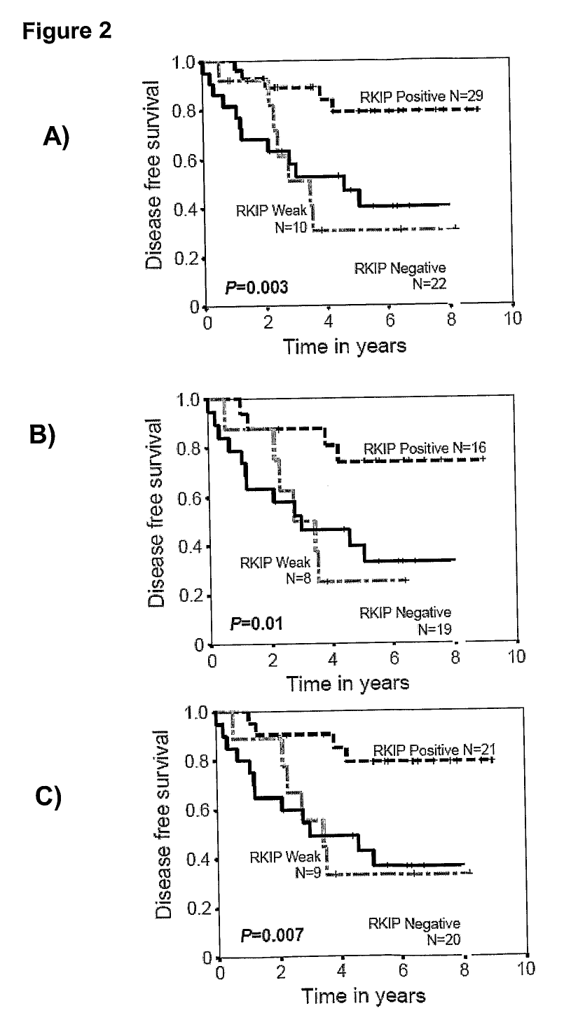

FIG. 2 shows Kaplan-Meier plots of disease-free survival of patients with early-stage colorectal cancers in relation to RKIP expression. (A) Effect of RKIP expression on Disease-Free Survival in 61 patients without stratification. (B) Data limited to Dukes' stage B1. (C) Data limited to patients who received surgery alone (Dukes' A and B1). Solid black lines represent RKIP negative patients, dashed black lines RKIP positive patients and gray dashed lines patients with weak RKIP expression. P values represent the Log-rank test.

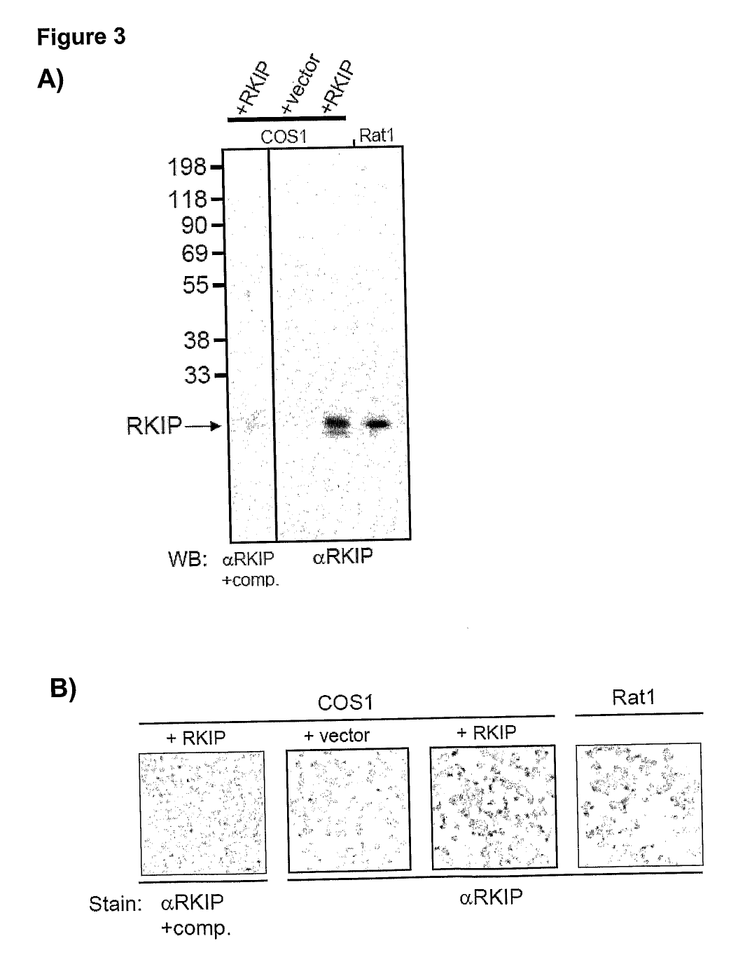

FIG. 3 shows Expression of RKIP in normal colon. (A) Specificity of the RKIP antibody. Lysates of cell lines expressing RKIP at very low (COS) or high (Rat1) levels were Western blotted with RKIP antibody. As positive control COS1 cells transfected with an RKIP expression vector were used. As negative control the RKIP antibody was preabsorbed with recombinant purified RKIP protein (αRKIP+comp.) before use on a section of the same Western blot. (B) The cell lines described in (A) were embedded in paraffin in order to test the RKIP antibody under conditions of Immunohistochemical staining.

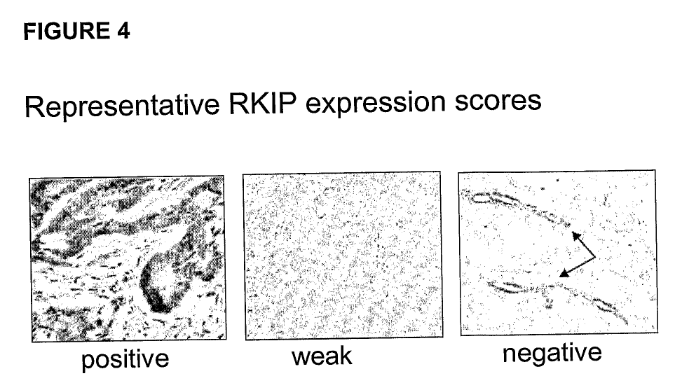

FIG. 4 shows scoring of RKIP expression in colon tumours, in an embodiment of the invention. Representative examples of tumours scored positive, weak positive and negative for RKIP are shown. The arrows indicate the strong RKIP staining of the endothelial lining of lymphatic vessels.

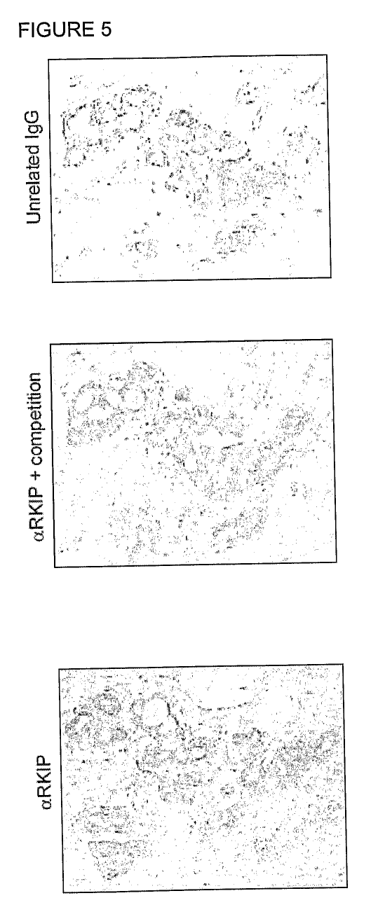

FIG. 5 shows specificity of the RKIP antibody. A breast lobule section was stained with unrelated IgG, unrelated antibody that had been preadsorbed with recombinant RKIP protein (+competition) or RKIP antibody. Magnification 200×.

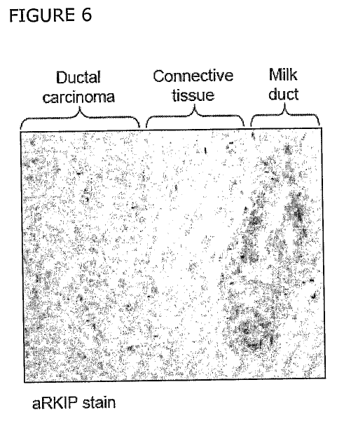

FIG. 6 shows RKIP stain of a section through a mammary ductal adenocarcinoma showing RKIP expression in the milk duct epithelial cells, connective tissue and tumour. Magnification 200×.

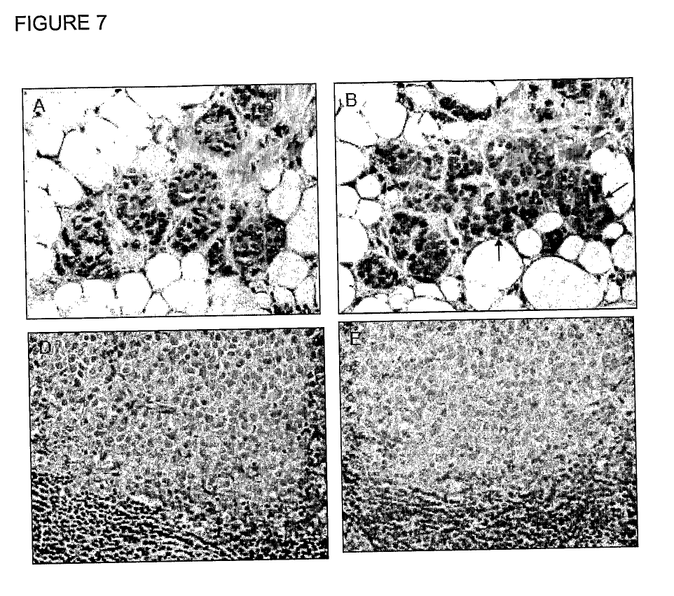

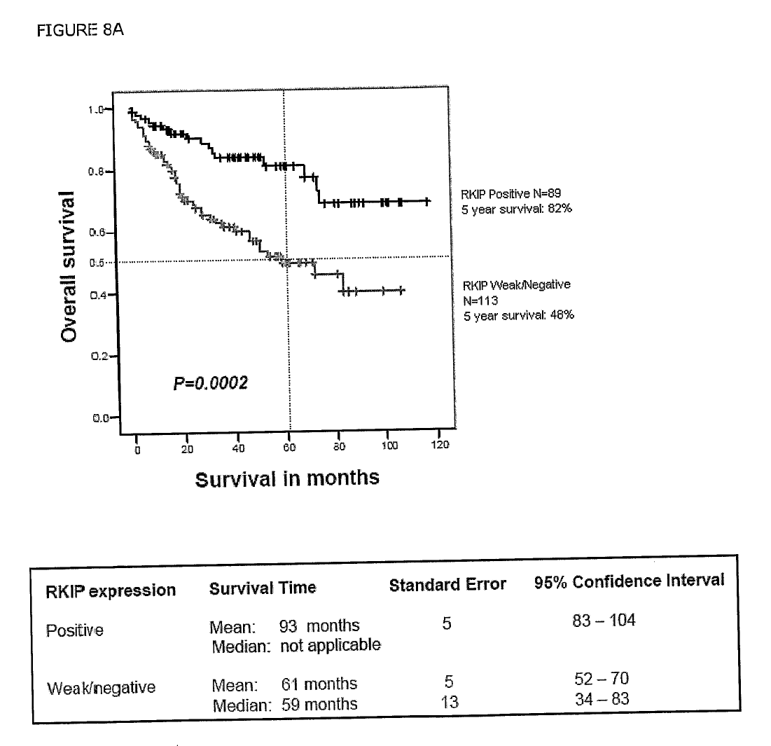

FIG. 7 shows RKIP expression in breast cancer. Haematoxylin and eosin stain of a node positive breast carcinoma (A) and the matched lymph-node metastasis (D). Immunohistochemical staining shows moderate RKIP immunoreactivity in primary tumour (B) compared to faint RKIP staining in the lymph node metastasis (E). Magnification 200×.

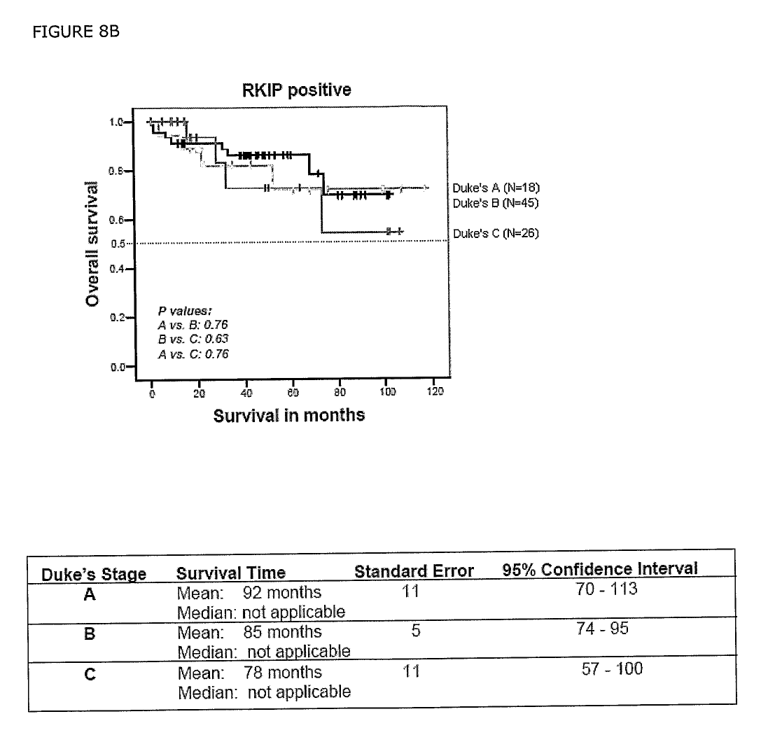

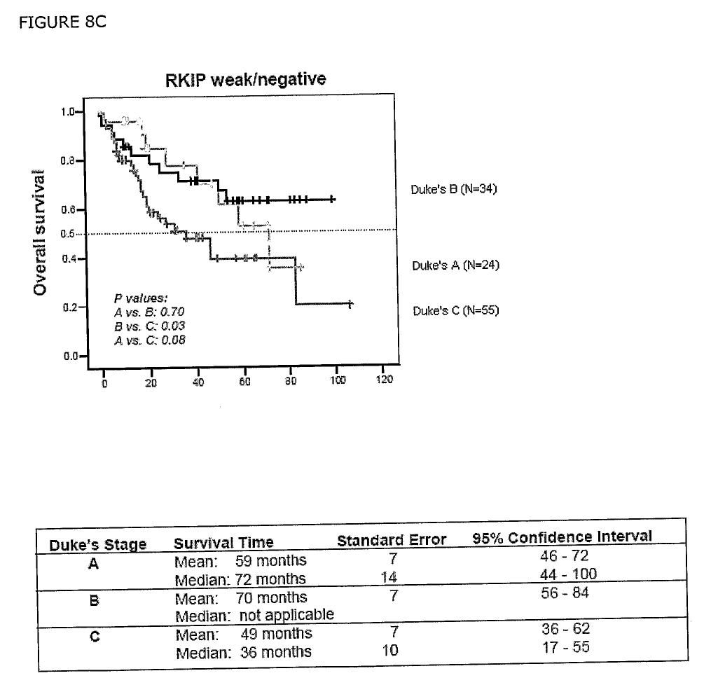

FIG. 8 shows the relationship between RKIP expression and survival in a sample of 268 patients.

### DETAILED DESCRIPTION OF THE INVENTION

### RKIP

RKIP is the Raf Kinase Inhibitor Protein. It was isolated as a protein which interferes with the activation of the Raf-MEK-ERK pathway by inhibiting the phosphorylation and activation of MEK by Raf-1 (Yeung et al, 2000; Yeung et al 1999b).

Reference to Raf Kinase Inhibitor protein herein may refer to any mammalian RKIP, e.g., rodent (rat, mouse, rabbit etc.) or primate, and preferably refers to human RKIP. A human RKIP sequence is given in gi|2135907|pir∥I53745.

| | |
|---|---|
| 1 | mpvdlskwsg plslqevdeq pqhplhvtya gaavdelgkv ltptqvknrp tsiswdglds |
| 61 | gklytlvltd pdapsrkdpk yrewhhflvv nmkgndissg tvlsdyvgsg ppkgtglhry |
| 121 | vwlvyeqdrp lkcdepilsn rsgdhrgkfk vasfrkkyel rapvagtcyq aewddyvpkl |
| 181 | yeqlsgk |

Alleles, homologous and orthologs of this sequence are also included.

RKIP proteins may also be defined as proteins having an RKIP motif, characterised by a consensus amino acid sequence:

TLX₃DPD (Z) PX₃ (B) X₄EX₂HXₙYX₄PX₍₂₋₄₎ GXHR (O)
VX (Z) X₃Q

wherein the single letter amino acid code is in accordance with the IUB/IUPAC code, X may be any amino acid, Z indicates a hydrophobic amino acid residue, B indicates a negatively charged amino acid residue (D or E), O indicates an aromatic amino acid residue (Y or F) and n is an integer from about 10 to about 50. Proteins comprising a sequence with at least 70% identical, preferably 80%, 90%, 95% or 99% identical to the consensus sequence may also be considered as having an RKIP motif. RKIP proteins preferably have such a motif comprised within a β fold structure composed of two antiparallel β sheets within the molecule.

### Patients and Treatment Regimes

The patient may be a mammal, and is preferably a human.

The patient may have been assessed as not showing detectable metastasis. For example, assessment may have been made that the patient has no detectable metastasis at the time of treatment of the primary tumour, or at the time of assigning a treatment or monitoring regime. The level of RKIP expression and/or activity can be used to assess the risk that this patient will show detectable metastasis in the future (i.e., will develop detectable metastasis), particularly the risk of metastatic recurrence after treatment of the primary tumour, and/or to assign an appropriate treatment to that patient based on the risk.

Assessment as to whether or not the patient has detectable metastasis may be done by any of the methods routine in the art. For example, the assessment may be done by physical examination, imaging techniques, radiology, histopathology, immunohistochemistry, RT-PCR or the like. Chest x-ray can be used to detect metastasis to the lungs. A CT (computerised tomography) scan can be used to detect metastasis to the lymph nodes, liver or lungs. Also, high levels of CEA or CA19-9 in the serum prior to the operation can indicate the presence of metastasis. The assessment may be carried out on the lymph nodes, or on other tissues. An absence of detectable metastasis may refer to an absence of detectable metastasis in the lymph nodes. It may refer to an absence of histopathologically detectable metastasis. In solid tumours that metastasise to local lymph nodes, such as colorectal cancer and breast cancer, the presence or absence of metastasis is preferably diagnosed by histological examination of lymph nodes removed during surgery.

The patient has preferably been diagnosed as having a non-metastatic primary tumour, or an early stage cancer. For example, the patient has preferably been diagnosed as having Dukes' A or B stage colorectal cancer, or an equivalent stage under another staging system. (Staging systems look at the tumour and the extent to which it has spread to other parts of the body to evaluate the progress of the cancer in the patient).

The Modified Dukes' staging system, which is well known in the art and which may be used herein is summarised below.

### Modified Dukes' Staging System

Modified Dukes' A The tumour penetrates into the mucosa of the bowel wall but no further.
Modified Dukes' B B1: tumour penetrates through the muscularis propria (the muscular layer) of the bowel wall but not throw the serosa.
   B2: tumour penetrates into and through the muscularis propria and serosa of the bowel wall.
Modified Dukes' C C1: Any invasive stage; there is pathologic evidence of colon cancer in the lymph nodes.
   C2: Any invasive stage; there is pathologic evidence of colon cancer in the Apical lymph nodes.
Modified Dukes' D The tumour has spread to organs such as the liver, lung or bone.

Other staging systems refer to stages I-IV, where stage I corresponds to Dukes' A, stage II to Dukes' B, stage III to Dukes' C and stage IV to Dukes' D. An example of a general staging system used for cancers is the TNM staging system. In this scores are assigned as below (exemplified particularly for colorectal cancer):

### Tumour:

T1: tumour invades submucosa;
T2: tumour invades muscularis propria;
T3: tumour invades through the muscularis propria into the subserosa or into the pericolic or perirectal tissues;
T4: tumour directly invades other organs or structures and/or perforates.

### Node;

N0: no regional lymph node metastasis;
N1: metastasis in 1 to 3 regional lymph nodes
N2: metastasis in 4 or more regional lymph nodes.

### Metastasis

M0: no distant metastasis
M1: distant metastasis present

### Stage Groupings:

### Stage I: T1 N0 M0; T2 N0 M0.

### Stage II: T3 N0 M0; T4 N0 M0.

### Stage III: any T, N1-2, M0;

### Stage IV: any T, any N, M1.

As stated above, stages I and II correspond to Dukes' A and B, for colorectal cancer.

In at least some aspects, the invention is concerned with metastatic relapse after treatment of the primary tumour. Thus, the patient may have had treatment of the primary tumour, e.g., by chemotherapy or radiotherapy, or preferably by surgery. The treatment of the primary tumour may have occurred at approximately the same time as the sample of the primary tumour is taken for determination of RKIP expression and/or activity. The sample may be obtained by biopsy, or may be obtained from the bulk tumour removed during surgery. If the latter, then the sample is considered to have been obtained at the same time as treatment of the primary tumour. If the sample is obtained by biopsy then it may be preferred that this biopsy is taken no more than a month before the treatment of the primary tumour, more preferably no more than a week or a day before.

Where the method comprises assigning a treatment and/or monitoring regime to be applied to the patient, this is preferably a regime to be applied in addition to the treatment of the primary tumour, e.g., an adjuvant treatment. The regime may be applied to reduce the risk of detectable metastasis developing and/or to detect such metastases as they do develop, in a patient who is not known to have detectable metastases at the time the regime is assigned. It may be a regime to be applied after treatment of the primary tumour, e.g., after removal of the primary tumour by surgery. It will be apparent that reference to a cancer patient need not suggest that the patient has evidence of a tumour at the time of the application of the treatment and/or monitoring regime, since this treatment and/or monitoring regime is preferably applied after treatment of the primary tumour and for the purpose of preventing or detecting relapse.

Where the method comprises identifying the level of risk of metastasis in a CRC patient, this is preferably the risk of metastatic recurrence after treatment of the primary tumour.

The step of taking a sample from the patient is not a required part of the present methods. Measuring the RKIP expression in a sample of primary tumour obtained from the patient does not comprise the step of obtaining the sample. In some embodiments of the invention, the method may include the additional step of taking a sample from the patient.

In at least some aspects and in embodiments, the method of the invention comprises assigning a treatment and/or monitoring regime. Assigning the regime is not intended to mean that the regime is actually applied, though in some embodiments of the invention, having assigned the regime the method may include the further step of applying the regime. The treatment regime may include for example chemotherapy, radiotherapy, immunotherapy, gene therapy, hormone therapy and/or surgery. As explained above, said regime may be a regime applied in addition to and preferably after treatment of the primary tumour, e.g., by removal, and before detection of metastasis (e.g., when no detectable disease is present). The regime may comprise early adjuvant chemotherapy.

The regime may additionally or alternatively comprise monitoring, particularly monitoring for metastatic recurrence. This may be by imaging (chest X-ray or CT scans, for example), fecal occult blood tests, double-contrast barium enema sigmoidoscopy, colonoscopy, digital rectal examination, biopsy, physical examination, blood tests and/or assessment of markers, particularly CEA, LASA, and/or CA19-9 or genetic testing for deletions in the APC and beta-catenin genes.

In the methods of the present invention, a lower RKIP expression and/or activity may indicate a higher level of risk, and/or result in assignment of a more rigorous treatment regime. This may for example comprise application of treatment (as compared to no treatment with good or high RKIP expression or the routine), application of more intensive or frequent treatment (as compared to high RKIP expression and/or to the routine treatment), and/or application of more frequent, extensive and/or intrusive monitoring (as compared to a good RKIP expression and/or to the routine treatment).

For example, low, e.g., weak or no RKIP expression may prompt close monitoring, e.g., a high-frequency monitoring regime, such as monitoring at a frequency of or greater than once every 3 months, preferably of or greater than once every two months. A close monitoring regime may also involve monitoring of CEA and/or CA19-9 levels and/or PET (positron emission tomography) SCAN. PET SCAN is an expensive monitoring technique not routinely used due to the cost, and targeted use for high risk patients would be beneficial.

Alternatively or additionally, low, e.g., weak or no RKIP expression may prompt the application of adjuvant treatment such as adjuvant chemotherapy, where none is routinely used.

The regime may also be more rigorous as a result of the combination of the monitoring and treatment, e.g., a monitoring regime with adjuvant treatment such as adjuvant chemotherapy applied on detection of signs of metastasis, wherein the threshold at which treatment is applied is lower than the routine threshold.

Classes indicating higher levels of RKIP expression, e.g., moderate or good RKIP expression, may result in assignment of a less rigorous regime to the patient, e.g., a regime in line with the routine regime for the cancer. This may for example mean a monitoring frequency of once every 6-12 months or longer, (and may mean a yearly monitoring) and/or no application of adjuvant chemotherapy following treatment of the primary tumour.

Where classification of or classifying RKIP expression levels is referred to herein (or other grammatical equivalents), this is preferably classification or classifying according to the risk of developing detectable metastases, e.g., in order to allow assignment of a treatment and/or monitoring regime.

Classification may be based on previously obtained expression levels of RKIP, e.g., in tumour samples. For example it may be done by comparison to a set of references based on previously obtained expression levels of RKIP. (The term “set” as used herein is intended to include the possibility of a single reference as well as a plurality of references).

The references may for example be levels detected in reference samples (i.e., the method may comprise comparing the level detected to a level detected in a reference sample). For example, where the method comprises detecting RKIP expression in situ, the reference may be a section from the sample, e.g., a stained section. The reference sample may be a sample from a normal tissue, from the same or a different individual. It may be a sample of tumour tissue, e.g., primary tumour or metastasised tumour. Where the sample is from a primary tumour, it may be a node negative or a node positive primary tumour. It may be a primary tumour from a patient who is known to have subsequently experienced metastatic relapse, and/or from a patient whose is known not to have subsequently experienced metastatic relapse, e.g., within five or ten years or longer. The tissue type of the sample is preferably the same as the tissue type of the primary tumour in the patient. Preferably, the sample is of colorectal tissue, e.g., a colorectal tumour. The set of references may include samples from a plurality of different tissues types (e.g., node negative and node positive primary tumours, from primary tumours showing or not showing metastatic relapse) and/or from a plurality of different patients having the same tissue type. These patients may for example have been assessed as showing typical expression and/or activity levels for groups at high or low risk of metastasis, or as showing boundary levels above or below which the sample should be considered as low or high risk.

In another embodiment, the references may be levels determined by examining samples from a plurality of individuals having primary tumours, and setting the reference based on observations made on this plurality of individuals. For example, the references may be established by: determining the level of RKIP expression in a sample from primary tumours from a plurality of patients who have been assessed as having no detectable metastasis; determining whether said patients develop detectable metastasis, and establishing a set of references which maximise the difference in probability of developing detectable metastasis between patients assigned to each class. This could be an ongoing process, that is, the reference could be subject to revision as further data is obtained. The references may for example express the bounds of each class which best distinguish between classes, e.g., between high and low risk patients. The boundaries may be expressed in numerical terms.

In a preferred embodiment, classification of the RKIP expression level in the sample comprises two steps: a first step of assigning a score to the sample based on the level of expression and/or activity, and a second step of translating that score into a class which indicates risk, e.g., “high” or “low” risk. The score may for example be based on the percentage of cells expressing RKIP or the intensity of the expression, and is preferably based on both. Methods of scoring have for example been described in Umemoto et al, 2001 and these methods are incorporated by reference herein. An example of such a method is given in the examples, in which a score is established corresponding to the sum of: [1] the percentage of positive cells; 1, <25% positive cells; 2, 26-50% positive cells and 3, >50% positive cells, and [2] the staining intensity (0, negative, 1, weak; 2, moderate; 3 strong). The set of references may comprise exemplary samples against which the staining intensity and/or the percentage of positive cells can be assessed by comparison. After attributing a score, the method may then comprise translating this into a grade. The set of references may therefore also or alternatively comprise the scores which form the bounds of each risk class. For example, in the examples given above, scores of 4 or less may be considered to correspond to RKIP expression which is weak or negative, and to a high risk of metastatic relapse, as compared to scores of 5 or 6 which may be considered to show RKIP positive tumours and to indicate a low risk of metastatic relapse.

Alternatively or additionally, the method may include comparison of the RKIP expression level to a control, e.g., an internal control. The skilled person would readily be able to select such a control. For example, a positive control may be a section of cells/tissue which tends to express RKIP at high levels, such as Auerbach's intramuscular plexuses or neuroendocrine crypt cells, normal epithelial mucosa or endothelial cells of blood or lymphatic vessels. A negative control may be a sample of tissue which has not been contacted with an RKIP detection agent. Other suitable controls may be readily identified by the person skilled in the art in the light of the information provided herein.

### Determining RKIP Expression Level

The sample obtained from the primary tumour may be a section of the primary tumour. The assessment of the level of an RKIP expression product may be carried on in situ on that section. For example, the assessment may be carried out by staining of the sample. In another embodiment the sample may be partially or fully lysed cells from the primary tumour.

In the present application, reference to determining the level of RKIP expression comprises determining the level of any RKIP gene expression product. RKIP gene expression products include protein and mRNA.

The level of RKIP protein can be indirectly assessed by activity levels of the protein. This may comprise contacting the sample with one or more components of the pathway in which RKIP acts. For instance, the method may comprise contacting the sample with a system which in the absence of RKIP permits phosphorylation of a substrate by an RKIP sensitive kinase, and determining the reduction of phosphorylation that occurs in the presence of the sample. An example of an RKIP sensitive kinase is Raf kinase, and an example of a substrate is MEK. ATP may also be included in the system. Another example of an RKIP sensitive kinase is IKKβ whose activity can be assayed by its ability to phosphorylate IκB in the presence of ATP. One of the components may be labelled. For example, the phosphate or the substrate may be labelled.

However, it is preferred that the level of an RKIP expression product (protein or mRNA) in the sample is assessed directly, e.g., by using a specific binding partner for the expression product.

Determination of protein or transcript level may be made by any of the methods known in the art. Determination may comprise measuring the level of expression product.

For example, suitable methods for assessing protein levels include immunohistochemistry (e.g., immunostaining, immunofluorescence), western blotting, and solid phase methods such as ELISA (enzyme-linked immunoabsorbant assay).

Using immunohistochemical techniques, an assessment of protein level can be made by determining the proportion of cells showing labelling and/or the intensity of the labelling (e.g., staining or fluorescence).

Transcript levels may be determined by in situ hybridisation, e.g., accompanied by assessment of the proportion of cells showing hybridisation and/or intensity of labelling.

Alternatively, or in addition, quantitative PCR methods may be used, e.g. based upon the ABI TaqMan™ technology, which is widely used in the art. It is described in a number of prior art publications, for example reference may be made to WO00/05409. PCR methods require a primer pair which target opposite strands of the target gene at a suitable distance apart (typically 50 to 300 bases). Suitable target sequences for the primers may be determined by reference to Genbank sequences.

Where many different gene transcripts are being examined, a convenient method is by hybridisation of the sample (either directly or after generation of cDNA or cRNA) to a gene chip array.

Where gene chip technology is used, the genes may be present in commercially available chips from Affymetrix, and these chips may be used in accordance with protocols from the manufacturer. Generally, methods for the provision of microarrays and their use may also be found in, for example, WO84/01031, WO88/1058, WO89/01157, WO93/8472, WO95/18376/WO95/18377, WO95/24649 and EP-A-0373203 and reference may also be made to this and other literature in the art.

The specific binding partner for a protein may be an antibody, as defined below, and is preferably a monoclonal antibody. The antibody may be detectably labelled.

Where the gene expression product is a transcript, the specific binding partner may be a nucleic acid sequence capable of specifically hybridising to said transcript. The nucleic acid sequence may be detectably labelled. It may be a primer, e.g., for quantitative PCR.

By “specific” is meant a binding partner which is suitable for detection of the transcript or protein in a complex mixture. The binding partner may bind to the gene expression product preferentially over other transcripts/proteins in the same species and may have no or substantially no binding affinity for other proteins or transcripts. In the case of a transcript, the transcript is preferably capable of distinguishing the target transcript from other transcripts in the mixture at least under stringent hybridisation conditions.

A label may be a radioactive, fluorescent chemiluminescent or enzyme label. Radioactive labels can be detected using a scintillation counter or other radiation counting device, fluorescent labels using a laser and confocal microscope, and enzyme labels by the action of an enzyme label on a substrate, typically to produce a colour change. After the binding reaction and any necessary separation step has taken place, the result of the assay is obtained by contacting the enzyme with a substrate on which it can act to produce an observable result such as a colour change, the extent of which depends on the amount of analyte originally in the sample. Suitable enzyme labels may give rise to detectable changes such as calorimetric, fluorometric, chemiluminescent or electrochemical changes, and include horseradish peroxidase and alkaline phosphatase, as well as lysozyme (detectable for example by lysis of organisms such as microccocus lysodeikticus), chymotrypsin, and E. coli DNA polymerase.

Other possible labels include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors.

Labelling may be indirect. For example, labelling may use a secondary, labelled binding partner, which is capable of binding to the complex between the RKIP expression product and the binding partner for that RKIP expression product. For example, the secondary binding partner may be a secondary antibody.

Other methods may also be used to detect interaction between the protein and the antibody, including physical methods such as surface plasmon resonance, agglutination, light scattering or other means.

In various aspects, the invention relates to kits which comprise a specific binding partner for a gene expression product. In some embodiments, the specific binding partner may be immobilised on a solid support.

Where the specific binding partner is an antibody, the kit may further comprise a detectably labelled moiety capable of binding to a complex between the protein and its specific binding partner. Additionally or alternatively, the kit may include one or more of the following reagents:
a) a reagent to fix a tissue, such as paraformaldeheyde;
b) a reagent to “unmask” cellular antigens upon fixation (such as EDTA-based solutions or citrate buffer); and/or
c) a detection system to reveal the enzymatic activity coupled to the primary antibody or the secondary moiety (e.g., secondary antibody), where the label is an enzyme, such as peroxidase.

For example, the kit may be for immunohistochemical techniques, and may comprise a first antibody capable of binding the protein to be detected, and a second, labelled antibody capable of binding said first antibody.

Alternatively, the kit may comprise a first, immobilised antibody capable of binding the protein to be detected and a second, labelled antibody capable of binding the protein when bound to the first antibody.

A kit according to the invention may alternatively or additionally comprise components for detecting RKIP activity, i.e., may comprise a system which in the absence of RKIP permits phosphorylation of a substrate by an RKIP sensitive kinase, as described above.

Methods of producing antibodies are known in the art. Preferred antibodies are isolated, in the sense of being free from contaminants such as antibodies able to bind other polypeptides and/or free of serum components. Monoclonal antibodies are preferred for some purposes, though polyclonal antibodies are within the scope of the present invention.

Where the kits comprise more than one antibody, these are preferably mixtures of isolated antibodies as described above.

Antibodies may be obtained using techniques which are standard in the art. Methods of producing antibodies include immunising a mammal (e.g. mouse, rat, rabbit) with a polypeptide of the invention. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992).

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies according to the present invention may be modified in a number of ways. Indeed the term “antibody” should be construed as covering any binding substance having a binding domain with the required specificity, e.g., antibody fragments, derivatives, functional equivalents and homologues of antibodies, including synthetic molecules and molecules whose shape mimics that of an antibody enabling it to bind an antigen or epitope.

Example antibody fragments, capable of binding an antigen or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Humanised antibodies in which CDRs from a non-human source are grafted onto human framework regions, typically with the alteration of some of the framework amino acid residues, to provide antibodies which are less immunogenic than the parent non-human antibodies, are also included within the present invention.

All documents referred to herein are incorporated in full by reference.

It will be apparent that the embodiments described herein are to illustrate the invention and that other embodiments are possible within the scope of the invention and will be apparent to the skilled person.

### EXAMPLES

The following examples are provided by way of illustration.

### Experimental Procedures

### RKIP Antibody and Western Blotting

RKIP antibody was obtained by immunising rabbits with purified recombinant RKIP protein produced in E. coli (Yeung et al., 1999a). For competition experiments the antibody was incubated with purified GST-RKIP protein bound to glutathione sepharose beads (Pharmacia) at an approximate 1:5 weight/weight ratio overnight. As control the antibody was incubated with GST protein beads. The beads were pelleted by centrifugation and the antibody supernatant was removed and used for Western blotting and immunohistochemistry, as described below. Western blotting was carried out as previously described (Yeung et al., 1999a).

### Immunohistochemistry

Formalin-fixed paraffin-embedded tissues were cut into 5 μm sections. Sections were deparafinised followed by rehydration through three different concentrations of alcohol, exposed to 0.3% H2O2 for 30 minutes to block endogenous peroxidase, and then rinsed with water. Epitope retrieval was carried out in 0.01 M EDTA solution at 95° C. for 20 minutes. Non-specific binding was blocked using 10% goat serum for 30 minutes. RKIP antibody was then applied to the sections at 1:500 dilution and incubated overnight at 4° C.

After 3 washes in PBS, secondary biotinylated goat anti-rabbit antibody was added for 30 minutes followed by streptavidin conjugated to horseradish peroxidase (dilution 1:500) for a further 30 minutes (Dako, C.A USA). The sections were then developed in 3,3-diaminobenzidine solution (DAB), counter stained with haematoxylin, dehydrated and mounted. The myenteric Auerbach's intramuscular plexuses and neuroendocrine crypt cells, which exhibit strong RKIP expression, were used as internal positive controls in all cases. For negative controls, the primary antibodies were substituted with goat serum or RKIP depleted antibody prepared as described above. Scoring of the antibodies stain was performed as previously described (Umemoto et al., 2001). The scoring method used ensures accurate representation of the areas and intensities of the stains. A score was established corresponding to the sum of: [1] the percentage of positive cells; 1, <25 ? positive cells; 2, 26-50% positive cells, and 3, >50% positive cells. [2] the staining intensity (0, negative; 1, weak; 2, moderate; 3, strong). The sum for the assigned values of the positive cell percentage [1] and the staining intensity [2] was 6 or less than 6. Scores between 0 and 2 were regarded as negative, scores of 3 and 4 as weakly positive, and scores of 5 and 6 as positive. All investigators performing the immunohistochemistry and the scoring were blinded to the clinical data.

### Apoptosis Assay

The Roche in situ cell death detection kit was used to detect apoptotic cells according to the manufacturer's protocol (Roche, Germany). In brief, 5 μm sections were deparaffinized as described above and treated with 20 μg/ml proteinase K diluted in 10 mM Tris-Hcl, pH7.5, for 10 minutes at room temperature. After 3 washes in PBS, the endogenous peroxidase was inactivated by incubation in 3% H2O2 in methanol for 20 minutes at room temperature.

After rinsing with deionised water, 50 μl of TUNEL reaction mixture was added to each slide. After incubation for 1 hour at 37° C. in a humidifying chamber slides were rinsed in PBS and 50 μl converter-POD (peroxidase) was applied for 30 minutes at room temperature. Slides were rinsed 3-times with PBS and colour development was initiated by the addition of 100 μl DAB-substrate solution. The reaction was terminated by dipping in distilled water. Slides were then rinsed in PBS and counterstained with haematoxylin. After dehydration by passing though graded alcohol and xylene, the slides were mounted using DPX mount. Apoptotic counts were taken from marked and random microscopic fields using a cell analysis system (CAS-200, Becton Dickinson, Elmhurst, Ill., U.S.A.) as previously described (Francis et al., 2000). The resultant count was also confirmed manually by counting apoptotic cells in 10-random-high power fields. The apoptotic index was the number of TUNEL positive nuclei per 1000 nuclei.

### Tissue Arrays

The specimens used to create each Landmark High Density Cancer Tissue Microarray (Ambion, Austin, USA) were derived from standard formalin-fixed, paraffin-embedded tissue blocks. Each slide contained a total of 280 samples comprising over 200 samples from 100-150 subjects with 1-2 specimens per subject and additional “matched” normal specimens where available. A separate control section of clinically “unmatched” normal specimens was included in the array. In addition, 45 early (non-metastatic) Dukes' A and B and 23 metastatic, Dukes' C and D primary colorectal cancers were included to improve on the quality of the statistical data obtained for the colorectal cancers contained in the tissue microarray.

### Statistical Analysis

Two-sided Chi-square or Fisher's exact tests were used for significance testing of contingency tables. Mann-Whitney U or Kruskal-Wallis tests were used to compare means.

Follow-up-to event outcomes were analyzed by Kaplan-Meier survival curves and compared by Log-rank tests. Univariate and multivariate Cox's proportional hazards models were used to analyse the effect of clinical characteristics on the survival of patients. All samples were scored independently by two pathologists. The strength of agreement between both pathologists' results was calculated using linear weighted kappa and was found to correlate well (κ>0.7). The primary outcomes were overall and disease-free survival. Overall survival is the time from study entry until death. Disease free survival is the time from study entry to first confirmed metastatic relapse.

All reported p-values are two-sided. Data were analyzed using the SPSS v11.0 software package.

For assessing disease-free survival, patients who died of causes unrelated to cancer but had no evidence of metastatic recurrence at the time of death were censored.

All work with patients' specimens and clinical data was carried out following the ethical guidelines and approval procedures implemented at the Universities of Glasgow, Aberdeen and Kuwait.

### Results and Discussion

We assayed RKIP expression in human tissues at the protein level. The antibody used was a rabbit polyclonal antiserum raised against a purified recombinant RKIP protein produced in E. coli (Yeung et al., 1999a). The specificity of this antibody was rigorously validated in both western blot and immunohistochemistry experiments (FIG. 1A and FIG. 3). On western blots the antibody exclusively detected RKIP in crude lysates of Rat1 cells, which are known to express high levels of RKIP protein (Yeung et al., 1999a). In contrast, no bands were detected in lysates of COS1 cells, which express very little RKIP (Yeung et al., 1999a). RKIP protein was only detected when COS1 cells were transfected with an RKIP expression vector. The reactivity of the RKIP antibody was substantially reduced by pre-adsorption with purified recombinant RKIP protein. Identical results were obtained when the antibody was used for immunohistochemistry using paraffin embedded COS1 and Rat1 cells (FIG. 3). Most importantly, pre-adsorbing the RKIP antibody with cognate antigen severely reduced its reactivity on paraffin sections of human tissues (FIG. 1A), demonstrating that the antibody specifically recognised RKIP protein, and thus could be used to examine the expression of RKIP in paraffin embedded human tissues.

In order to survey the expression of RKIP protein in humans we employed a tissue array featuring 280 samples taken from tumours and matched normal tissues where available (Table 1). The tissues investigated included breast, respiratory tract (mouth, larynx, pharynx, lung), genitourinary system (ovary, prostate, bladder, kidney), digestive tract (salivary glands, stomach, pancreas, liver, gallbladder, colon). RKIP protein was low in connective tissue and lymphocytes. In contrast, RKIP was highly expressed in epithelial and endothelial cells in all tissues including glandular epithelia of breast, pancreas and salivary glands, tubular epithelia and glomeruli of the kidney, transition epithelia of the bladder, and both lymphatic as well as blood vessel endothelia. High RKIP expression also was observed in neuronal cells, hepatocytes and muscle cells. Tumours derived from these tissues exhibited variable RKIP protein levels, often with a trend towards reduced RKIP expression. In tumours of the kidney and pancreas this trend of reduced RKIP, expression came close to statistical significance with p-values of 0.053 and 0.054, respectively, suggesting that the level of RKIP expression decreases during tumour formation. In colon cancer the reduction of RKIP levels was statistically significant with a p-value of 0.008 (Table 1).

TABLE 1
RKIP expression in human tissues and cancers.

| | Patients | RKIP expression status | | | | | | |
|---|---|---|---|---|---|---|---|---|
| Tissue Types | No. | Negative | | Weak | | Positive | | P* |
| Bladder | 17 | | | | | | | |
| Normal bladder | 1 | 0 | (0%) | 0 | (0%) | 1 | (100%) | NS |
| Non-metastatic bladder cancer | 15 | 1 | (6.7%) | 2 | (13.3%) | 12 | (80%) | |
| Metastatic bladder cancer | 1 | 0 | (0%) | 0 | (0%) | 1 | (100%) | |
| Brain | 9 | | | | | | | |
| Normal brain | 3 | 0 | (0%) | 0 | (0%) | 3 | (100%) | NS |
| Brain tumours | 6 | 0 | (0%) | 0 | (0%) | 6 | (100%) | |
| Breast | 19 | | | | | | | |
| Normal breast | 6 | 0 | (0%) | 0 | (0%) | 6 | (100%) | NS |
| Non-metastatic breast cancer | 3 | 0 | (0%) | 0 | (0%) | 3 | (100%) | |
| Metastatic breast cancer | 10 | 0 | (0%) | 4 | (40%) | 6 | (60%) | |
| Colon | 75 | | | | | | | |
| Normal colon | 6 | 0 | (0%) | 0 | (0%) | 6 | (100%) | 0.008 |
| Non-metastatic colon cancer | 44 | 10 | (22.7%) | 7 | (15.9%) | 27 | (61.4%) | |
| Metastatic colon cancer | 25 | 13 | (52%) | 5 | (20%) | 7 | (28%) | |
| Stomach | 5 | | | | | | | |
| Normal Gastric mucosa | 2 | 0 | (0%) | 0 | (0%) | 2 | (100%) | NS |
| Non-metastatic gastric cancer | 1 | 0 | (0%) | 1 | (100%) | 0 | (0%) | |
| Metastatic gastric cancer | 2 | 0 | (0%) | 1 | (50%) | 1 | (50%) | |
| Kidney | 28 | | | | | | | |
| Normal kidney | 5 | 0 | (0%) | 0 | (0%) | 5 | (100%) | NS |
| Kidney cancer | 23 | 2 | (8.7%) | 13 | (56.5%) | 8 | (34.8%) | (p = 0.053) |
| Liver/gallbladder | 7 | | | | | | | |
| Normal | 4 | 0 | (0%) | 0 | (0%) | 4 | (100%) | NS |
| Non-metastatic cancer | 3 | 1 | (33.3%) | 1 | (33.3%) | 1 | (33.3%) | |
| Lung | 14 | | | | | | | |
| Normal lung | 3 | 0 | (0%) | 3 | (100%) | 0 | (0%) | NS |
| Non-metastatic lung cancer | 8 | 1 | (12.5%) | 2 | (25%) | 5 | (62.5%) | |
| Metastatic lung cancer | 3 | 0 | (0%) | 1 | (33.3%) | 2 | (66.7%) | |
| Ovary | 12 | | | | | | | |
| Non-metastatic ovarian cancer | 11 | 0 | (0%) | 2 | (18%) | 9 | (82%) | NS |
| Metastatic ovarian cancer | 1 | 1 | (100%) | 0 | (0%) | 0 | (0%) | |
| Pancreas | 15 | | | | | | | |
| Normal pancreas | 7 | 0 | (0%) | 1 | (14.3%) | 6 | (85.7%) | NS |
| Non-metastatic pancreatic cancer | 8 | 2 | (25%) | 4 | (50%) | 2 | (25%) | (p = 0.054) |
| Metastatic pancreatic cancer | 0 | 0 | (0%) | 0 | (0%) | 0 | (0%) | |
| Prostate | 19 | | | | | | | |
| Normal prostate | 6 | 0 | (0%) | 0 | (0%) | 6 | (100%) | NS |
| Non-metastatic prostate | 13 | 0 | (0%) | 4 | (30.8%) | 9 | (69.2%) | |
| Salivary/parotid glands | 6 | | | | | | | |
| Non-metastatic cancer | 5 | 0 | (0%) | 0 | (0%) | 5 | (100%) | NS |
| Metastatic Parotid cancer | 1 | 0 | (0%) | 0 | (0%) | 1 | (100%) | |
| Squamous epithelium | 31 | | | | | | | |
| Normal tissue/Tongue, lips, | 7 | 0 | (0%) | 1 | (14.3%) | 6 | (85.7%) | NS |
| mouth, larynx and pharynx | | | | | | | | |
| Non-metastatic squamous cancer | 22 | 0 | (0%) | 2 | (9.1%) | 20 | (90.9%) | |
| Metastatic squamous cancer | 2 | 0 | (0%) | 0 | (0%) | 2 | (100%) | |

The Landmark High Density Cancer Tissue Microarray (Ambion, Austin, USA) was stained with RKIP antibody.
*P-values were calculated using two-sided Fisher's exact tests. NS, not significant

We examined the expression of RKIP in normal colon in more detail. The colonic epithelium is replenished regularly from stem cells dividing in the crypts. Cells differentiate as they move upwards out of the crypts. RKIP expression is almost undetectable in the crypts, but steadily increases as the cells differentiate (FIG. 1B). This is similar to the pattern of RKIP expression observed in the skin, where RKIP expression is barely observed in the basal layers, but increases as cells differentiate (Schuierer et al., 2004; Yamazaki et al., 2004). RKIP also was found highly expressed in Auerbach's plexus (FIG. 1C). These are ganglions of the myoenteric nerve cell system embedded in the muscular layer of the colon. Furthermore, the RKIP antibody strongly stained a few individual cells in the crypts. Co-staining with chromogranin A identified them as neuroendocrine cells (FIG. 1D). These cells are the source of numerous gastrointestinal neuropeptides that regulate gastrointestinal physiology, but also can function as autocrine growth factors for colon cancers (Moody et al., 2003).

For the analysis of RKIP expression in colon tumours we firstly examined samples from 88 patients from patients with different stages of sporadic colon cancers graded according to the Dukes' system.

All work with patients' material and clinical data was carried out according to the ethical guidelines implemented at the University of Glasgow. A total of 88 patients with sporadic colorectal cancers were examined in this study: 23 patients with Dukes' C and D stage cancer; 23 patients with early colorectal cancer who had no evidence of metastatic disease at the time of surgery, but subsequently relapsed with metastasis; 28 matched patients who remained disease-free after surgery; and 14 Dukes' B2 staged patients who were treated surgically and with 6 cycles of standard chemotherapy and were followed-up prospectively. All 65 patients with Dukes' A and B stage cancers were followed-up at regular intervals for a minimum period of 2 years (range: 2 to 9 years) and were clinically assessed for symptoms and signs of recurrence. Metastatic recurrences were confirmed radiologically and/or histologically or at postmortem. For assessing disease-free survival, patients who died of causes unrelated to cancer but had no evidence of metastatic recurrence at the time of death were censored. Four patients were lost to follow-up and 10 patients had no recurrence time recorded but died from metastatic disease. For these patients the date of death was used to calculate the disease free-survival.

The Dukes' system classifies colon cancers as stage A when they are confined to the epithelial layer, and stage B when they show penetration through the muscular layer (Dukes' B1) or the serosa (Dukes' B2). Both stages A and B do not exhibit detectable metastases. Dukes' C tumours correspond to stage B tumours with lymph node metastases, while Dukes' D tumours have metastasised to distant sites beyond the lymph nodes. As shown in Table 2 the 23 patients with Dukes' C and D tumours exhibited a highly significant reduction or loss of RKIP expression in their primary tumours (p=0.009).

TABLE 2
Clinical characteristics of patients in relation to RKIP expression

| | | Patients with | Patients with | Patients with | |
|---|---|---|---|---|---|
| Clinical | Patients | no RKIP | weak RKIP | positive RKIP | P |
| Characteristics | (N = 88) | expression | expression | expression | values* |
| Dukes' stage and treatment | | | | | |
| A and B1 (Surgery only) | 51 | 20 (39%) | 9 (18%) | 22 (43%) | 0.198^{b} |
| B2 (Surgery and chemotherapy) | 14 | 2 (14%) | 4 (29%) | 8 (57%) | |
| C and D | 23 | 13 (56%) | 5 (22%) | 5 (22%) | 0.009^{b} |

*P values were calculated using two-sided Fisher's exact tests^{b}.

We also conducted a larger trial, using a TMA prepared from 268 CRC patients selected from the Aberdeen Colorectal Tumour Bank (Table 3).

TABLE 3
Clinical Data of the Aberdeen CRC patient cohort

| Patient population | | |
|---|---|---|
| Gender | 268 | (100%) |
| Male | 135 | (50.4%) |
| Female | 133 | (49.6%) |
| Age | | |
| Mean | 68.29 | yrs |
| Range | 33-92 | yrs |
| <70 yrs | 127 | (47.4%) |
| >70 yrs | 141 | (52.6%) |
| Dukes' stage | 268 | (100%) |
| A | 53 | (19.8%) |
| B | 104 | (38.8%) |
| C | 111 | (41.4%) |
| Tumour site | 268 | (100%) |
| Proximal colon | 95 | (35.4%) |
| Distal colon | 97 | (36.2%) |
| Rectum | 76 | (28.4%) |
| Tumour differentiation | 268 | (100%) |
| Well | 10 | (3.7%) |
| Moderate | 228 | (85.1%) |
| Poor | 30 | (11.2%) |

All these patients had undergone surgery for CRC at Aberdeen Royal Infirmary between 1994 and 2003. The TMA was produced as described previously (Dundas et al 2005), Kumarakulasingham et al (2005).

In order to assess RKIP expression in CRC a TMA containing samples from 268 patients with Dukes' A (19.8%), B (38.8%), and C (41.4%) CRC was stained for RKIP protein (Table 4). We excluded samples that detached during staining, showed great intra-tumour heterogeneity of RKIP expression, or contained mostly non-tumour tissue. As expected from previous studies (Chatterjee et al 2004, Fu et al 2003), RKIP expression was downregulated in lymph nodes metastases. Of 79 lymph node metastases on the array 67 (85%) had no or weak RKIP expression compared to 12 (15%) that expressed RKIP, confirming that the metastatic process in CRC also involves a reduction or loss of RKIP protein expression. More interestingly, however, 202 primary tumour samples eligible to analysis showed a highly significant positive correlation (p=0.0002) between RKIP expression and overall survival (FIG. 8A). Patients whose CRC scored positive for RKIP expression had a mean survival time of 93 months, whereas low or negative RKIP expression correlated with a shortened mean survival time of 61 months. As the survival curve of RKIP positive patients never fell under 50%, no median survival could be calculated. RKIP expression was independent of p53 status, tumour differentiation, tumour site (proximal colon, distal colon, rectum) and B-raf expression according to multivariate Cox's proportional hazards model (Table 4).

TABLE 4
Cox proportion Hazard analysis.

| | B | SE | Wald | df | Sig. | Hazard ratio |
|---|---|---|---|---|---|---|
| Dukes' Stage | | | | | | |
| Dukes' A (42; 19.8%) vs. B (81; 38.2%) | −.519 | .343 | 2.287 | 1 | .130 | .595 |
| Dukes' B (81; 38.2%) vs. C (89; 42.0%) | −.871 | .301 | 8.388 | 1 | .004 | .419 |
| Tumour Site | | | | | | |
| Proximal (78; 36.8%) vs. distal (78; 36.8%) | −.191 | .353 | .292 | 1 | .589 | .826 |
| Rectal (56; 26.4%) vs. distal (78; 36.8%) | .386 | .320 | 1.462 | 1 | .227 | 1.472 |
| Differentiation | | | | | | |
| Moderate (178; 84.0%) vs. well (10; 4.7%) | .579 | .723 | .641 | 1 | .423 | 1.784 |
| Poor (24; 11.3%) vs. moderate (178; 84.0%) | .985 | .772 | 1.625 | 1 | .202 | 2.677 |
| RKIP expression | | | | | | |
| Weak & negative (113; 63.2%) vs. positive (89; 36.8%) | 1.045 | .331 | 9.970 | 1 | .002 | 2.843 |
| p53 expression | | | | | | |
| Weak (76; 35.8%) vs. positive (78; 36.8%) | −.611 | .389 | 2.466 | 1 | .116 | .543 |
| Negative (58; 27.4%) vs. positive (78; 36.8%) | .235 | .266 | .782 | 1 | .377 | 1.265 |
| B-Raf expression | | | | | | |
| Weak (76; 35.8%) vs. positive (78; 36.8%) | .077 | .328 | .054 | 1 | .816 | 1.080 |
| Negative (58; 27.4%) vs. positive (78; 36.8%) | −.267 | .417 | .410 | 1 | .522 | .766 |

The number of patients evaluated and percentage is given in parenthesis.

This analysis further demonstrated that negative or weak RKIP expression is associated with a significant hazard ratio of 2.84, which is comparable to the risk associated with an advanced Dukes' stage. As Dukes' staging is still the clinical gold standard for CRC risk prediction, we stratified the cohort according to Dukes' stage and RKIP expression. This analysis showed that within Dukes' stage C tumours, patients whose CRC were positive for RKIP expression had a mean survival of 78 months, which is statistically not significantly different from the overall survival of patients with RKIP positive Dukes' A and B tumours with mean survival periods of 92 and 85 months, respectively (FIG. 8B). Vice versa, weak or negative RKIP expression reduced the overall survival in all Dukes' stages (FIG. 8C). For instance, the mean survival of Dukes' A versus Dukes' B and Dukes' C patients was 59, 70 and 49 months, respectively. These data suggested that RKIP expression in the primary tumour can predict patients' overall survival independent of Dukes' stage and could be useful as prognostic marker to assess the risk of metastatic relapse.

These results suggested that RKIP expression in the primary tumour may be associated with the occurrence of metastasis. As 10-45% of Dukes' A and B patients relapse with metastatic disease (Compton, 2003; Kahlenberg et al., 2003) we investigated whether RKIP expression in the primary tumour could predict the risk of metastasis in Dukes' A and B patients.

Therefore, we analyzed the expression of RKIP protein in paraffin-embedded tissue sections from 65 early stage CRC patients where primary clinical and follow-up data were available. Of these patients 37 were males and 28 were females. The mean age was 65 years at the time of diagnosis. 17 patients had right-sided colon cancer and 43 had cancer of the left colon including 17 patients with cancer of the rectum. Seven patients were Dukes' stage A, 44 were Dukes' stage B1 and 14 were Dukes' stage B2. All patients were treated with surgery. The 14 Dukes' stage B2 patients in addition received 6 courses of chemotherapy consisting of the standard regimen of 5-Fluorouracil in combination with Leucovorin after surgical resection (table 5). All patients were followed up for a minimum of 2 years (range: 2 to 9 years), and metastatic recurrences were confirmed radiologically, histologically or postmortem. For assessing disease-free survival patients who died of causes unrelated to cancer, but had no evidence of metastatic recurrence at the time of death, were censored. Four patients were lost to follow-up and 10 patients had no recurrence time recorded, but died from metastatic disease. For these patients the date of death was used to calculate the disease free-survival. 40 patients remained disease-free. 25 patients with no evidence of metastasis at the time of surgery subsequently relapsed with metastases.

TABLE 5
Clinical characteristics of patients in relation to RKIP expression

| | | Patients with | | Patients with | | Patients with | | |
|---|---|---|---|---|---|---|---|---|
| Clinical | | no RKIP | | weak RKIP | | positive RKIP | | P |
| Characteristics | Patients | expression | | expression | | expression | | values* |
| Sex | | | | | | | | |
| Male | 37 | 12 | (32%) | 6 | (16%) | 19 | (51%) | 0.558^{a} |
| Female | 28 | 10 | (36%) | 7 | (25%) | 11 | (39%) | |
| Age | | | | | | | | |
| Mean in years | 65 | 66 | | 68 | | 65 | | 0.469^{c} |
| Site† | | | | | | | | |
| Right sided | 17 | 5 | (29%) | 4 | (24%) | 8 | (47%) | 0.755^{b} |
| Left sided | 43 | 16 | (37%) | 7 | (16%) | 20 | (47%) | |
| Differentiation ‡ | | | | | | | | |
| Well | 25 | 13 | (52%) | 5 | (20%) | 7 | (28%) | 0.003^{b} |
| Moderate | 31 | 6 | (19%) | 4 | (13%) | 21 | (68%) | |
| Poor | 7 | 2 | (29%) | 4 | (57%) | 1 | (14%) | |
| Counts Mean | | | | | | | | |
| Mitosis (10 high-power fields) | 5.74 | 6.5 | | 5.3 | | 5.3 | | 0.498^{c} |
| Apoptosis/1000 nuclei | 11.9 | 6.3 | | 11.3 | | 15 | | 0.024^{c} |
| pT stage§ | | | | | | | | |
| pT1 and pT2 | 24 | 8 | (33%) | 4 | (17%) | 12 | (50%) | 0.889^{b} |
| pT3 and pT4 | 37 | 13 | (35%) | 8 | (22%) | 16 | (43%) | |
| Lymphatic invasion | | | | | | | | |
| Yes | 12 | 2 | (17%) | 2 | (17%) | 8 | (67%) | 0.283^{b} |
| No | 53 | 20 | (38%) | 11 | (21%) | 22 | (42%) | |
| Vascular invasion | | | | | | | | |
| Yes | 11 | 1 | (9%) | 2 | (18%) | 8 | (73%) | 0.131^{b} |
| No | 54 | 21 | (39%) | 11 | (20%) | 22 | (41%) | |
| Metastatic recurrence | | | | | | | | |
| Yes | 25 | 13 | (52%) | 7 | (28%) | 5 | (20%) | 0.004^{a} |
| No | 40 | 9 | (23%) | 6 | (15%) | 25 | (63%) | |
| % 5-Years disease free | | | | | | | | |
| survival (95% CI) | | | | | | | | |
| All patients | 61 | 47 | (25.2-69.1) | 31 | (1.9-59.6) | 79 | (62.9-95.9) | |
| Surgery only | 50 | 43 | (20.4-65.5) | 33 | (2.5-64.1) | 79 | (60.8-97.5) | |
| Surgery and chemotherapy | 11 | NA** | | 0 | | 85.71 | (59.8-111) | |
| Median survival in years | | | | | | | | |
| (Standard error) | | | | | | | | |

| | | | | | | | |
|---|---|---|---|---|---|---|---|
| All patients | 61 | 4.57 | (1.41) | 3.46 | (0.81) | >8 | 0.004^{d} |
| Surgery only | 50 | 3.01 | (1.65) | 3.46 | (1.06) | >8 | 0.006^{d} |

| | | | | | |
|---|---|---|---|---|---|
| Surgery and chemotherapy | 11 | NA** | 2.4 | >8 | 0.234^{d} |

*P values were calculated using two-sided Chi Square^{a} or Fisher's exact tests^{b}. Kruskal-Wallis^{c} test was used to compare means. Log-rank test^{d} was used to compare survival data. NS, not significant
†Cancer site was colonic but unknown side in 5 cases. Right sided cancers include caecum, ascending colon. Left-sided cancers include transverse, descending, sigmoid colon and rectum
‡Differentiation was undetermined in 2 cases
§T stage could not be assessed in 4 cases
**For the patients with no RKIP expression and treated with Surgery and Chemotherapy the Survival
Estimates cannot be computed since all observations are censored (no recurrences).

TABLE 6
Clinical characteristics of patients in
relation to disease- recurrence status

| | | Metastatic | | Cancer did | | |
|---|---|---|---|---|---|---|
| Clinical | Patients | recurrence | | not recur | | |
| Characteristics | (N = 65) | (N = 25) | | (N = 40) | | P values* |
| Sex | | | | | | |
| Male | 37 | 11 | (30%) | 26 | (70%) | 0.096^{a} |
| Female | 28 | 14 | (50%) | 14 | (50%) | |
| Age | | | | | | |
| Mean in years | 65 | 69 | | 63 | | 0.105^{c} |
| Site† | | | | | | |
| Right sided | 17 | 6 | (35%) | 11 | (65%) | 0.640^{a} |
| Left sided | 43 | 18 | (42%) | 25 | (58%) | |
| Differentiation ‡ | | | | | | |
| Well | 25 | 13 | (52%) | 12 | (48%) | 0.083^{a} |
| Moderate | 31 | 8 | (26%) | 23 | (74%) | |
| Poor | 7 | 4 | (57%) | 3 | (43%) | |
| Counts Mean | | | | | | |
| Mitosis/(10 high- | 5.74 | 6.2 | | 5.4 | | 0.087^{c} |
| power fields) | | | | | | |
| Apoptosis/1000 | 11.9 | 10 | | 12.5 | | 0.339^{c} |
| nuclei | | | | | | |
| Dukes' stage and | | | | | | |
| treatment | | | | | | |
| A and B1 (Surgery | 51 | 23 | (45%) | 28 | (55%) | 0.036^{a} |
| only) | | | | | | |
| B2 (Surgery and | 14 | 2 | (14%) | 12 | (86%) | |
| chemotherapy) | | | | | | |
| pT stage§ | | | | | | |
| pT1 and pT2 | 24 | 9 | (38%) | 15 | (63%) | 0.979^{a} |
| pT3 and pT4 | 37 | 14 | (38%) | 23 | (62%) | |
| Lymphatic invasion | | | | | | |
| Yes | 12 | 4 | (33%) | 8 | (67%) | 0.754^{b} |
| No | 53 | 21 | (40%) | 32 | (60%) | |
| Vascular invasion | | | | | | |
| Yes | 11 | 2 | (18%) | 9 | (82%) | 0.181^{b} |
| No | 54 | 23 | (43%) | 31 | (57%) | |

*P values were calculated using two-sided Chi-square^{a} or Fisher's exact tests^{b}. Mann-Whitney U^{c} test was used to compare means.
†Cancer site was colonic but unknown side in 5 cases. Right sided cancers include caecum, ascending colon. Left-sided cancers include transverse, descending, sigmoid colon and rectum.
‡Differentiation was undetermined in 2 cases.
§T stage could not be assessed in 4 cases.

Multivariate Cox's proportional hazards model was used to assess the influence of RKIP expression and the clinical characteristics of the patients on their survival. When the patients with metastatic recurrence were matched with patients who remained disease-free, no significant association was observed between sex, age and tumour site with respect to metastatic recurrence. Similarly, tumour differentiation, mitotic and apoptotic indexes, lymphatic and vascular invasion, and the depth of invasion did not significantly correlate with metastatic relapse (table 6). These results confirm the limited usefulness of current clinico-pathological parameters in identifying patients at risk from metastatic relapse in early CRC.

In contrast, RKIP expression exhibited a strong and statistically highly significant correlation with disease free survival in Dukes' stage A and B patients (Table 5). Of the 25 patients with metastatic recurrence, 13 (52%) had lost RKIP expression, 7 (28%) were weakly positive and only 5 (20%) expressed RKIP in their primary tumours. The median survival for patients who were treated with surgery alone and lacked RKIP expression was 4.57 years, compared to 3.46 years with weak RKIP expression and more than 8 years in patients with positive RKIP expression. Similarly, five years disease-free survival of patients treated with surgery alone was: 47% (95% CI 20.4-65.5) for patients with RKIP negative tumours, 31% (95% CI 2.5-64.1) for patients with weakly positive and 79% (95% CI 60.8-97.5) for patients with RKIP positive tumours. The median survival of patients with RKIP negative or weakly positive tumours was 4.57 and 3.46 years, respectively. In contrast, patients with RKIP positive tumours survived more than 8 years (p=0.004). The striking difference in disease free survival dependent on the expression of RKIP becomes even more evident when displayed as Kaplan-Meier plot (FIG. 2). Patients with RKIP positive tumours had stable disease free survival rates at around 90% between years 2 and 4 after surgery, and at around 80% from year 4 onwards. In contrast, patients with reduced or no RKIP expression experienced a steady decline in disease-free survival time during the whole observation period. This pattern was similar in the non-stratified patient population (p=0.003), in Dukes' B1 patients (p=0.01), and in patients treated with surgery alone (p=0.007). For comparison, the expression of RKIP in the primary tumours of 25 CRC patients with metastatic disease at presentation (Dukes' stage C and D) was also very significantly reduced with 13 patients showing loss of RKIP expression, five patients exhibiting weak, and seven patients positive expression (Table 1). These data demonstrate that the level of RKIP expression in primary CRCs is significantly and inversely associated with metastatic disease. Importantly, in CRC patients with non-metastatic disease RKIP expression in the primary tumour predicts the risk of metastasis development. The strong correlation between RKIP expression and metastatic recurrence was found to be independent of other parameters and clinical characteristics including sex, age, tumour sites, mitotic index, lymphatic or vascular invasions, Dukes' and pT-stages (table 6). This independence was statistically significant (p=0.039) demonstrating that the predictive value of RKIP expression as a marker for metastatic recurrence is independent of other risk factors.

To investigate the association of metastatic phenotype and loss or reduced RKIP expression further, we also examined biological and molecular parameters that have been show to be of prognostic value in other cancer types. These included mitotic index, tumour differentiation, VEGF production, tumour blood vessel counts, p53 expression and apoptotic index in the primary tumours of our patient cohort. There was no significant association found between these parameters and RKIP expression (data not shown) with two exceptions, apoptotic index and tumour differentiation (Table 2). There was a significant association between RKIP expression and tumour differentiation (p=0.003). This result is in keeping with the observation that RKIP expression increases as colon epithelial cells differentiate (FIG. 1B) (although this difference was not significant in the larger trial using data from the Aberdeen Colorectal Tumour Bank) reported above). In addition, the apoptotic index, which represents the number of apoptotic cells per 1000 nuclei, was significantly associated with RKIP status (p=0.024). Tumours with negative or weak RKIP expression had significantly lower apoptotic indexes (mean apoptotic indexes 6.3 and 11.3, respectively) than tumours expressing RKIP (mean apoptotic index 15).

In summary, our results show a significant relationship between reduced RKIP expression, metastatic recurrence and reduced disease-free survival in patients with early colorectal cancers after surgical treatment. Of note, 5 Dukes' B2 patients who received adjuvant therapy did not relapse, although their tumours expressed no or weak RKIP. This result may suggest a benefit of chemotherapy for patients whose tumours weakly express RKIP.

Thus, our study identifies RKIP as a single and independent marker protein in the primary tumour that could predict the risk of early CRC to metastasize, and hence guide the therapeutic strategy. This finding gains in significance given the high prevalence of CRC and the urgent demand for molecular markers that can predict the main determinant for the prognosis and treatment disease, i.e. metastasis.

### Breast Cancer

Information is also provided below on work relating to breast cancer.

### Materials and Methods

### Patient Samples

Human breast cancer samples were obtained from 103 patients, including 52 node-negative patients and 51 node-positive patients (with 51 paired lymph node metastases), following partial or total mastectomy, with informed consent about the use of resected tumours for research purposes according to the guidelines for research on human tissue samples set by the University of Glasgow. Parallel samples were processed for histological examination by haematoxylin and eosin staining.

Prognostic indicators of tumour size, grade, histological type and estrogen receptor status were subsequently assessed by a breast cancer pathologist.

### Immunohistochemistry

Sections of formalin-fixed, paraffin-embedded tissue (5 μM) were deparafinised in two changes of histoclear, and rehydrated through graded alcohols to distilled water.

Antigen retrieval was performed using 0.01M EDTA buffer pH 8.0 for RKIP, and 0.01M citrate buffer pH 6.0 for phospho-ERK at 95° C. for 20 minutes. RKIP protein expression was examined using a 1:1500 dilution of polyclonal rabbit antibody raised against a recombinant full length RKIP protein. Antibody binding was detected using the streptavidin-biotin method (Vector ABC Elite Detection Kit, Peterborough, UK) and 3,3-diaminobenzidine (DAB) as chromogen (Vector DAB substrate kit). Slides were counterstained with hematoxylin, dehydrated and mounted. Paraffin-embedded sections of prostate cancer served as positive controls for RKIP expression, and omission of the primary antibody as negative control. The specificity of the RKIP antibody was assured by (i) replacing it with unrelated IgG, and (ii) preadsorbing the RKIP antibody with an excess of cognate antigen (recombinant purified RKIP produced in E. coli) before use (FIG. 5).

In order to quantify tumour cell apoptosis, a subset of tumours from each group were stained using the In Situ Cell Death Detection Kit (Roche Diagnostics Ltd, Bell Lane, Lewes, UK), according to the manufacturer's instructions. Briefly, paraffin-embedded sections were dewaxed, rehydrated to water and incubated with 20 μg/ml proteinase K for 20 minutes at room temperature. Endogenous peroxidase activity was quenched with 1% H202 and the TUNEL reaction mixture was applied to sections for 1 hour at 37° C. Following several PBS washes, slides were incubated with 50 μl converter peroxidase for 30 minutes. DNA breakpoints were visualized using DAB, which stained apoptotic cell nuclei dark brown.

### Evaluation of Immunostaining

All slides were examined and scored by a breast pathologist, who was blinded to both clinical and pathological data. RKIP expression levels were scored using the visual grading system into four classes, whereby 0=negative, 1=weak, 2=moderate and 3=intense staining. No score 3 was assigned to RKIP staining in breast cancer samples.

### Statistical Analysis

Statistical analysis was performed using the SPSS software. The Wilcoxon sum rank test was used for comparing RKIP expression in primary tumours with metastases, while the Mann-Whitney U-test was used to compare RKIP in node-negative to node-positive breast tumours.

### Results

### Breast Tissue Pathology

Histological typing of primary tumours identified ductal carcinoma as the predominant cancer type, with only occasional presence of other types, such as lobular (4), mucinous (1), cribriform (1) and tubular (3). Tumours ranged in size from 5-120 mm (node negative, mean=19.95 mm) and 6-70 mm (node-positive, mean=23.59 mm), and represented all three stages of poorly, moderately and well-differentiated tumours.

### RKIP Expression in Primary Breast Tumours

All of the primary breast tumours analyzed in this study demonstrated RKIP immunoreactivity, except for one node-negative tumour. RKIP protein was found to be predominantly cytoplasmic, although some nuclear staining was noted. RKIP expression was observed in normal epithelial cells of the milk duct, ductal carcinoma in situ (DCIS) and cancer cells. RKIP was not detectably expressed in the extracellular matrix or in the connective tissues of breast sections. The RKIP antibody was specific as an unrelated IgG control or RKIP antibody preadsorbed with recombinant RKIP protein produced in E. coli did not result in any staining (FIG. 5).

Intense RKIP staining was observed in normal milk duct epithelial cell, whereas RKIP expression was downregulated in primary breast carcinomas (FIG. 6).

RKIP expression was moderate in 31/51 (60.8%) node-positive tumours and weakly positive in 20/51 (39.2%) cases (FIG. 6). In the node-negative tumours, RKIP expression was moderate in 37/52 cases (71.2%), weakly positive in 14/52 cases (26.9%) and negative in 1/52 cases (1.9%). This distribution of RKIP downregulation is suggestive of a trend that primary tumours with reduced levels of RKIP expression have a higher tendency to metastasize (p=0.306).

### RKIP Expression is Significantly Diminished in Metastatic Breast Cancer

RKIP expression in the node-positive tumours was predominantly moderate in intensity. By contrast, in the matched lymph node metastases RKIP expression was considerably diminished (FIG. 7) and in a significant number of cases entirely absent ( 9/51, 17.7%). In total, cases (58.8%) were weakly positive for RKIP, and cases (23.5%) were moderately positive. This decrease of RKIP expression in metastases was found to be highly consistent and statistically significant (p=0.000003) (Table 7) suggesting that reduction of RKIP expression is a hallmark of metastatic disease in human breast cancer. No correlation was found between RKIP expression and established clinical and pathological breast cancer markers including histological type, tumour differentiation grade, size, or estrogen receptor status (data not shown), suggesting that RKIP is independent of other markers for breast cancer progression and prognosis.

TABLE 7
1a) RKIP expression in Node Positive versus Node negative Primary Tumours

| | | Node Positive | | Node Negative | | Total | |
|---|---|---|---|---|---|---|---|
| p = 0.306 | | number | % | number | % | number | % |
| RKIP | Negative or | 0 | 0 | 1 | 1.9 | 1 | 1 |
| expression | very faint | | | | | | |
| (primary | Faint | 20 | 39.2 | 14 | 26.9 | 34 | 33 |
| tumours) | Moderate | 31 | 60.8 | 37 | 71.2 | 68 | 66 |
| Total | | 51 | 100 | 52 | 100 | 103 | 100 |

1b) RKIP expression in Primary Tumours versus Lymph Node Metastases

| | | RKIP expression (primary tumours) | | | | | |
|---|---|---|---|---|---|---|---|
| | | Faint | | Moderate | | | |
| | | RKIP stain | | RKIP stain | | Total | |
| p = 0.000003 | | number | % | number | % | number | % |
| RKIP | Negative or | 6 | 66.7 | 3 | 33.3 | 9 | 17.6 |
| expression | very faint | | | | | | |
| (metastases) | Faint | 13 | 43.3 | 17 | 56.7 | 30 | 58.8 |
| | Moderate | 1 | 8.3 | 11 | 91.7 | 12 | 23.5 |
| Total | | 20 | 39.2 | 31 | 60.8 | 51 | 100 |

Table 7 (a) RKIP staining in the node-negative versus node-positive tumours. Node positive primary tumours show a statistically non-significant trend to reduced RKIP expression. Data were evaluated using Mann-Whitney U test. (b) RKIP expression is significantly reduced in lymph node metastases compared to primary tumours (p=0.000003). Data were evaluated using the Wilcoxon signed rank sum test.

### REFERENCES

Andre, T. and de Gramont, A. (2004). An overview of adjuvant systemic chemotherapy for colon cancer. Clin. Colorectal Cancer 4 Suppl 1:S22-8., S22-S28.
Barratt, P. L., Seymour, M. T., Stenning, S. P., Georgiades, I. Walker, C., Birbeck, K., and Quirke, P. (2002). DNA markers predicting benefit from adjuvant fluorouracil in patients with colon cancer: a molecular study. Lancet 360, 1381-91.
Buyse, M. and Piedbois, P. (2001). Should Dukes' B patients receive adjuvant therapy? A statistical perspective. Semin Oncol 28, 20-4.
Cancer Research UK (2004). Large Bowel Cancer Factsheet—April 2004. http://www.cancerresearchuk.org/aboutcancer/statistics/statsmisc/pdfs/factsheet_bowel_apr2004.pdf.
Chatterjee, D., Bai, Y., Wang, Z., Beach, S., Mott, S., Roy, R., Braastad, C., Sun, Y., Mukhopadhyay, A., Aggarwal, B. B., Darnowski, J., Pantazis, P., Wyche, J., Fu, Z., Kitagwa, Y., Keller, E. T., Sedivy, J. M., and Yeung, K. C. (2004). RKIP sensitizes prostate and breast cancer cells to drug-induced apoptosis. J. Biol Chem. 279, 17515-17523.
Compton, C. C. (2003). Colorectal carcinoma: diagnostic, prognostic, and molecular features. Mod. Pathol. 16, 376-388.
Dorudi, S., Hanby, A. M., Poulsom, R., Northover, J., and Hart, I. R. (1995). Level of expression of E-cadherin mRNA in colorectal cancer correlates with clinical outcome. Br J Cancer 71, 614-6.
Dundas S R, Lawrie L C, Rooney P H, Murray G I. Mortalin is over expressed by colorectal adenocarcinomas and correlates with poor survival. J Pathol 2005; 205:74-81.
Francis, I. M., Adeyanju, M. O., George, S. S., Junaid, T. A., and Luthra, U. K. (2000). Manual versus image analysis estimation of PCNA in breast carcinoma. Anal Quant Cytol Histol 22, 11-6.
Fu, Z., Smith, P. C., Zhang, L., Rubin, M. A., Dunn, R. L., Yao, Z., and Keller, E. T. (2003). Effects of raf kinase inhibitor protein expression on suppression of prostate cancer metastasis. J. Natl. Cancer Inst. 95, 878-889.
Greten, F. R. and Karin, M. (2004). The IKK/NF-kappaB activation pathway—a target for prevention and treatment of cancer. Cancer Lett. 206, 193-199.
Hartsough, M. T. and Steeg, P. S. (1998). Nm23-H1: genetic alterations and expression patterns in tumour metastasis. Am J Hum Genet 63, 6-10.
Heys, S. D., Langlois, N., Smith, I. C., Walker, L. G., and Eremin, O. (1998). NM23 gene product expression does not predict lymph node metastases or survival in young patients with colorectal cancer. Oncol Rep 5, 735-9.
Kahlenberg, M. S., Sullivan, J. M., Witmer, D. D., and Petrelli, N. J. (2003). Molecular prognostics in colorectal cancer. Surg. Oncol. 12, 173-186.
Kumarakulasingham M, Rooney P H, Dundas S R, Telfer C, Melvin W T, Curran S et al. Cytochrome p450 profile of colorectal cancer: identification of markers of prognosis. Clin Cancer Res 2005; 11:3758-65.
Liefers, G. J., Cleton-Jansen, A. M., van de Velde, C. J., Hermans, J., van Krieken, J. H., Cornelisse, C. J., and Tollenaar, R. A. (1998). Micrometastases and survival in stage II colorectal cancer. N Engl J Med 339, 223-8.
Lombardi, D. P., Geradts, J., Foley, J. F., Chiao, C., Lamb, P. W., and Barrett, J. C. (1999). Loss of KAI1 expression in the progression of colorectal cancer. Cancer Res 59, 5724-31.
Marsoni, S. (2001). Efficacy of adjuvant fluorouracil and leucovorin in stage B2 and C colon cancer. International Multicenter Pooled Analysis of Colon Cancer Trials Investigators. Semin Oncol 28, 14-9.
Moody, T. W., Chan, D., Fahrenkrug, J., and Jensen, R. T. (2003). Neuropeptides as autocrine growth factors in cancer cells. Curr. Pharm. Des 9, 495 509.
Mulder, J. W., Kruyt, P. M., Sewnath, M., Oosting, J., Seldenrijk, C. A., Weidema, W. F., Offerhaus, G. J., and Pals, S. T. (1994). Colorectal cancer prognosis and expression of exonv6-containing CD44 proteins. Lancet 344, 1470-2.
Olson, R. M., Perencevich, N. P., Malcolm, A. W., Chaffey, J. T., and Wilson, R. E. (1980). Patterns of recurrence following curative resection of adenocarcinoma of the colon and rectum. Cancer 45, 2969-74.
Ovaska, J., Jarvinen, H., Kujari, H., Perttila, I., and Mecklin, J. P. (1990). Follow-up of patients operated on for colorectal carcinoma. Am J Surg 159, 593-6.
Petersen, V. C., Baxter, K. J., Love, S. B., and Shepherd, N. A. (2002). Identification of objective pathological prognostic determinants and models of prognosis in Dukes' B colon cancer. Gut 51, 65-9.
Ragnhammar, P., Hafstrom, L., Nygren, P., and Glimelius, B. (2001). A systematic overview of chemotherapy effects in colorectal cancer. Acta Oncol. 40, 282-308.
Reddy, K. B., Nabha, S. M., and Atanaskova, N. (2003). Role of MAP kinase in tumour progression and invasion. Cancer Metastasis Rev. 22, 395-403.
Schuierer, M. M., Bataille, F., Hagan, S., Kolch, W., and Bosserhoff, A. K. (2004). Reduction in Raf kinase inhibitor protein expression is associated with increased Ras-extracellular signal-regulated kinase signaling in melanoma cell lines. Cancer Res 64, 5186-5192.
Umemoto, M., Yokoyama, Y., Sato, S., Tsuchida, S., Al Mulla, F., and Saito, Y. (2001). Carbonyl reductase as a significant predictor of survival and lymph node metastasis in epithelial ovarian cancer. Br J Cancer 85, 1032-6.
Yamaguchi, A., Urano, T., Goi, T., Saito, M., Takeuchi, K., Hirose, K., Nakagawara, G., Shiku, H., and Furukawa, K. (1996). Expression of a CD44 variant containing exons 8 to 10 is a useful independent factor for the prediction of prognosis in colorectal cancer patients. J. Clin. Oncol. 14, 1122-1127.
Yamazaki, T., Nakano, H., Hayakari, M., Tanaka, M., Mayama, J., and Tsuchida, S. (2004). Differentiation induction of human keratinocytes by phosphatidylethanolamine-binding protein. J. Biol Chem. 279, 32191-32195.
Yeung, K., Janosch, P., McFerran, B., Rose, D. W., Mischak, H., Sedivy, J. M., and Kolch, W. (2000). Mechanism of suppression of the Raf/MEK/Extracellular signal-regulated kinase pathway by the raf kinase inhibitor protein. Mol. Cell Biol. 20, 3079-3085.
Yeung, K., Seitz, T., Li, S., Janosch, P., McFerran, B., Kaiser, C., Fee, F., Katsanakis, K. D., Rose, D. W., Mischak, H., Sedivy, J. M., and Kolch, W. (1999a). Suppression of Raf-1 kinase activity and MAP kinase signalling by RKIP. Nature 401, 173-177.
Yeung, K., Seitz, T., Li, S., Janosch, P., McFerran, B., Kaiser, C., Fee, F., Katsanakis, K. D., Rose, D. W., Mischak, H., Sedivy, J. M., and Kolch, W. (1999b). Suppression of Raf-1 kinase activity and MAP kinase signalling by RKIP. Nature 401, 173-177.
Yeung, K. C., Rose, D. W., Dhillon, A. S., Yaros, D., Gustafsson, M., Chatterjee, D., McFerran, B., Wyche, J., Kolch, W., and Sedivy, J. M. (2001). Raf kinase inhibitor protein interacts with nf-kappab-inducing kinase and tak1 and inhibits nf-kappab activation. Mol. Cell Biol. 21, 7207-7217.
Zhou, W., Goodman, S. N., Galizia, G., Lieto, E., Ferraraccio, F., Pignatelli, C., Purdie, C. A., Piris, J., Morris, R., Harrison, D. J., Paty, P. B., Culliford, A., Romans, K. E., Montgomery, E. A., Choti, M. A., Kinzler, K. W., and Vogelstein, B. (2002). Counting alleles to predict recurrence of early-stage colorectal cancers. Lancet 359, 219-25.

1-25. (canceled)

26. A method of assigning a treatment and/or monitoring regime to a cancer patient, comprising:
determining the RKIP expression level in a sample from a primary tumour obtained from the patient;
assigning a treatment and/or monitoring regime to the patient according to the RKIP expression level.

27. The method of claim 26, wherein said regime is to be applied in addition to treatment of the primary tumour.

28. The method of claim 27, wherein said regime is to be applied to a patient assessed as not having detectable metastasis.

29. The method of claim 26, wherein the primary tumour is from the group selected from cancer of the lung, breast, prostate, pancreas, brain, bladder, stomach, kidney, liver, gallbladder, ovary, testes, cervix, uterus, salivary and parotid glands, squamous epithelium, skin, bone and neuronal cancer.

30. The method of claim 26, wherein the primary tumour is a colorectal tumour.

31. The method of claim 30, wherein the primary tumour is Duke's A or B stage colorectal cancer.

32. The method of claim 26, wherein the treatment regime is selected from the group consisting of chemotherapy, radiotherapy, immunotherapy, gene therapy, hormone therapy, surgery and a combination thereof.

33. The method of claim 26, wherein the treatment regime is an adjuvant treatment regime.

34. The method of claim 26, wherein determining the RKIP expression level comprises contacting a sample from the primary tumour with a specific binding partner of an RKIP expression product.

35. The method of claim 34, wherein the RKIP expression product is a protein.

36. The method of claim 34, wherein the binding partner for an RKIP expression product is an antibody against RKIP.

37. The method of claim 36, wherein the antibody is monoclonal.

38. The method of claim 34, wherein the RKIP expression product is an mRNA.

39. The method of claim 38, wherein the specific binding partner is a nucleic acid sequence capable of specifically hybridising to said mRNA.

40. The method of claim 39, wherein the specific binding partner is a primer.

41. The method of claim 34, wherein the binding partner is labelled.

42. A method of predicting metastasis in a cancer patient, comprising determining the RKIP expression level in a sample from a primary tumour obtained from the patient.

43. A method of classifying the expression level of RKIP in a sample from a primary tumour from a cancer patient in order to assign a treatment and/or monitoring regime based on said expression levels, the method comprising determining the level of expression of RKIP, and classifying the level of expression.

44. The method of claim 43, wherein the primary tumour is not associated with detectable metastases.

45. The method of claim 43, wherein the treatment regime is to be applied in addition to treatment of the primary tumour.

46. A kit for classifying the RKIP expression level in a sample of primary tumour obtained from a cancer patient in order to assign a treatment and/or monitoring regime based on said expression level, wherein said kit comprises a specific binding partner for an RKIP expression product and a set of references against which the expression level can be compared in order to classify said level.

47. A kit for classifying the RKIP expression level in a sample of primary tumour obtained from a cancer patient in order to assign a treatment and/or monitoring regime based on said expression level, wherein the kit comprises a specific binding partner for an RKIP expression product, and also comprises a specific binding partner for the expression product of a gene other than RKIP, wherein the expression levels of said gene are of value in prognosis of the cancer.

48. The kit of claim 46, wherein the RKIP expression product is a protein.

49. The kit of claim 46 wherein the binding partner for an RKIP expression product is an antibody against RKIP.

50. The kit of claim 49, wherein the antibody is monoclonal.

51. The kit of claim 46, wherein the RKIP expression product is an mRNA.

52. The kit of claim 51, wherein the specific binding partner is a nucleic acid sequence capable of specifically hybridising to said mRNA.

53. The method or kit of claim 52, wherein the specific binding partner is a primer.

54. The kit of claim 46, wherein the binding partner is labelled.

55. A method of establishing a set of references for classifying the RKIP expression level in a sample from a primary tumour in order to assign a treatment and/or monitoring regime based on the expression level, the method comprising:
determining the RKIP expression level in a sample from primary tumours obtained from a plurality of patients, wherein said patients have been assessed as having no detectable metastasis;
determining whether said patients experience metastatic relapse;
establishing a set of references for classifying the RKIP expression level which maximise the difference in risk of relapse between patients assigned to each class.

56. An apparatus for classifying the RKIP expression level in a sample from a primary tumour in order to assign a treatment and/or monitoring regime based on said expression level, the apparatus comprising:
a port for receiving the sample;
a detector for detecting the expression level of RKIP in said sample;
means for providing a set of references for classifying the RKIP expression level;
means for comparing the detected expression level to said references so as to classify said expression level;
an output for providing the classification of the sample to the user.
